(19) United States
(12) Patent Application Publication
(10) Pub. No.: US 2005/0250737 A1
(43) Pub. Date: Nov. 10, 2005

(54) Therapeutic ophthalmic compositions containing retinal friendly excipients and related methods

(75) Inventors: Patrick M. Hughes, Aliso Viejo, CA (US); Laurent Delahaye, Mougins (FR); Michele Boix, Mougins (FR); James N. Chang, Newport Beach, CA (US); Robert T. Lyons, Laguna Hills, CA (US)

Correspondence Address: STOUT, UXA, BUYAN & MULLINS LLP, 4 VENTURE, SUITE 300, IRVINE, CA, 92618, US

(73) Assignee: Allergan, Inc., Irvine, CA (US)

(21) Appl. No.: 11/091,977

(22) Filed: Mar. 28, 2005

Related U.S. Application Data
(63) Continuation-in-part of application No. 10/966,764, filed on Oct. 14, 2004.
(60) Provisional application No. 60/530,062, filed on Dec. 16, 2003.
(60) Provisional application No. 60/519,232, filed on Nov. 12, 2003.

(51) Int. Cl. A61K 31/724; A61K 31/573

(52) U.S. Cl. 514/58; 514/171

(57) ABSTRACT

Pharmaceutical compositions suitable for administration into the interior of an eye of a person or animal are described. The present compositions include one or more components which are effective in providing a reduced toxicity relative to existing intraocular ophthalmic compositions. The present compositions include one or more therapeutic agents in amounts effective in providing a desired therapeutic effect when placed in an eye, and one or more retinal friendly excipients that have a reduced toxicity relative to benzyl alcohol or polysorbate 80. In certain compositions, the excipient component of the compositions comprises one or more cyclodextrins or cyclodextrin derivatives. Methods of using the compositions to treat ocular conditions are also described.

### CROSS-REFERENCE TO RELATED APPLICATIONS

The present application is a continuation-in-part of U.S. application Ser. No. 10/966,764, filed Oct. 14, 2004, which claims the benefit of U.S. Application No. 60/530,062, filed Dec. 16, 2003, and U.S. Application No. 60/519,237, filed Nov. 12, 2003, the contents of which in their entireties are hereby incorporated by reference.

### BACKGROUND

The present invention relates to pharmaceutical compositions and methods for using such compositions to treat diseases or disorders of one or more eyes of an individual. More specifically, the present invention relates to ophthalmic compositions useful for administration to the interior of an eye of an individual to treat an ocular condition without causing substantial toxicity, damage, or injury to intraocular tissues.

The retinal pigmented epithelium (RPE) is made up of a monolayer of polarized cells attached on Brüch's membrane. The RPE sustains photoreceptor cell integrity and function through phagocytosis and regeneration of visual pigment, active transport of metabolites, light absorption, and maintenance of outer blood-retina barrier. Alterations in RPE cell functions can cause various pathologies of the retina. RPE phenotype changes are known to result in dysregulation of extracellular matrix synthesis and degradation. In addition, RPE cells play a critical role in the metabolism of the retina. RPE cells are responsible for the transport of nutrients to rod and cone photoreceptors and removal of waste products to the blood. RPE cells are part of the outer blood-retinal barrier which confers to the eye an immune privilege (Streilein J W et al., “Ocular immune privilege: therapeutic opportunities from an experiment of nature”, Nature Reviews Immunology, 2003, 3:879-89). Therefore, RPE cells are often the targeted cells for therapeutics for example to treat proliferative vitreoretinopathy (PVR) or angiogenesis defect-induced pathologies such as age-related macular degeneration (AMD).

In certain ocular conditions, the retina can change or become damaged and thereby negatively affect vision of an individual. For example, in ocular condition, such as dry age related macular degeneration (ARMD), lesions form beneath the macula due to RPE changes. These lesions, drusen, comprise lipid-rich extracellular matrix components and may coalesce overtime resulting in a shallow elevation of the RPE cells. The RPE cells begin to clump, aggregate, and atrophy. Degeneration of the RPE cells leads to a secondary degeneration of the overlying photoreceptors. Clearly, anything that can disrupt the RPE can dramatically affect vision.

Many existing therapies for ocular diseases and disorders utilize topical ophthalmic compositions. These treatments often require frequent administration of the topical ophthalmic compositions. Typically, less than 5% of a drug or therapeutic agent in topical eye drops reach anterior intraocular tissues. Reasons for low bioavailability include poor penetration across the corneal barrier and rapid loss of the instilled solution from the precorneal area. Very little drug further reaches the posterior segment of the eye; the retina, RPE, optic nerve head and vitreous. The amount reaching the retina from topical ocular dosing typically represents a million fold dilution. Hence, direct intraocular administration is required for many drugs targeting the posterior segment ocular tissues.

Cyclodextrins are cyclic oligosaccharides containing 6, 7, or 8 glucopyranose units, referred to as alpha-cyclodextrin, beta-cyclodextrin, or gamma-cyclodextrin, respectively. Cyclodextrins have been shown to increase aqueous solubility and chemical stability of numerous poorly water-soluble drugs, reduce local irritation, and often enhance bioavailability of the drug to ocular tissues. For example, see U.S. Pat. No. 4,727,064 (Pitha); U.S. Pat. No. 5,324,718 (Loftsson); U.S. Pat. No. 5,332,582 (Babcock et al.); U.S. Pat. No. 5,494,901 (Javitt et al.); U.S. Pat. No. 6,407,079 (Muller et al.); U.S. Pat. No. 6,723,353 (Beck et al.); and U.S. patent Publication Nos. 2002/0198174 (Lyons) and 2004/0152664 (Chang et al.); and Rao et al., “Preparation and evaluation of ocular inserts containing norfloxacin”, Turk J Med Sci, 2004, 34:239-246. Thus, cyclodextrins have been used to solubilize and/or stabilize therapeutic agents in topical ophthalmic compositions. However, complexes of a cyclodextrin and a drug do not appear to permeate the cornea.

More recently, intraocular ophthalmic compositions have been developed and utilized to treat ocular diseases and disorders. By administering a therapeutic agent directly into the eye, it is possible to address problems associated with topical administration of drugs.

As one example, among the therapies currently being practiced to treat ocular posterior segment disorders, such as uveitis, macular degeneration, macular edema and the like, intravitreal injection of a corticosteroid, such as triamcinolone acetonide has been employed. See, for example, U.S. Pat. No. 5,770,589 (Billson et al.). However, many compounds are known to be toxic to the retina, including pharmaceutically active agents, such as chloroquine and canthanxanthin. In addition to pharmacologically active compounds, an overlooked source of drug induced retinal toxicity includes drug formulation excipients. The importance of understanding retinal toxicity due to therapeutic agents and/or excipients present in ophthalmic compositions becomes clear when compositions are administered into the eye where the components of such compositions can directly interact with retinal cells and tissue.

Triamcinolone acetonide has received a lot of attention recently due to its efficacy in treating macular edema. Kenalog®-40 is a commercially available formulation of triamcinolone acetonide, approved for intramuscular and intraarticular administration. Kenalog®-40 is reconstituted and injected directly into the vitreous of an eye. Each milliliter (ml) of the Kenalog® 40 composition includes 40 milligrams (mg) of triamcinolone acetonide, sodium chloride as a tonicity agent, 10 mg of benzyl alcohol as a preservative, and 7.5 mg of carboxymethylcellulose and 0.4 mg of polysorbate 80 as resuspension aids.

Although widely used by ophthalmologists, this commercially available formulation suffers from several important limitations. After intravitreal injection, triamcinolone acetonide and all formulation excipients contact the RPE. The retina does not possess intercellular tight junctions and poses little resistance to molecules diffusing to the level of the RPE. Kenalog®-40 injection, when administered intravitreally, has been implicated in non-bacterial endophthalmitis.

The formulation excipients benzyl alcohol (preservative) and/or polysorbate 80 (surfactant) are thought to be the cause of non-bacterial endophthalmitis associated with intravitreal injection of Kenalog-40. For example, the presence of benzyl alcohol preservative and polysorbate 80 surfactant tends to lead to unnecessary and/or undue cell damage or other toxicities in ocular tissues. Even though some clinicians routinely “wash” the triamcinolone acetonide precipitate several times with saline to reduce the concentration of these undesirable materials, such washing is inconvenient, time consuming, and most importantly, increases the probability of microbial or endoxin contamination that could lead to intraocular infection and inflammation.

Moreover, the triamcinolone acetonide in the Kenalog® 40 tends to rapidly separate and precipitate from the remainder of the composition. For example, this composition, if left standing for 1 to 2 hours, results in a substantial separation of a triamcinolone acetonide precipitate from the remainder of the composition. Thus, if the composition is to be injected into the eye, it must be vigorously shaken and used promptly after being so shaken in order to provide a substantially uniform suspension in the eye. In addition, resuspension processing requires the use of the resuspension aids noted above, at least one of which is less than totally desirable for sensitive ocular tissues, such as the RPE.

Thus, there remains a need for new compositions and methods which may be used to treat ocular conditions by being intraocularly administered to a patient and which have little or no adverse reactions to the patient receiving the compositions.

### SUMMARY

The present invention addresses this need and provides pharmaceutical compositions and methods that provide effective treatment of one or more ocular conditions without causing substantial damage or injury to ocular tissues. Among other things, the present compositions may be administered into or in the vicinity of an eye of a patient with reduced inflammation resulting from administration of the composition, but not necessarily caused by the drug itself. The present compositions are useful for delivery to the interior of an eye of a individual, such as a person or animal. The compositions comprise a therapeutic component and an excipient component.

The therapeutic component is present in an amount effective in providing a desired therapeutic effect when administered to the interior of an eye, such as the posterior segment of an eye. The therapeutic component may comprise one or more agents selected from the group consisting of anti-angiogenic agents, anti-inflammatory agents, and neuroprotective agents, among others.

The excipient component of the present compositions may be present in an amount that is less toxic to RPE cells compared to excipients currently used in ophthalmic compositions. The excipient component may comprise one or more inert substances or agents, such as agents selected from the group consisting of viscosing agents (viscosity inducing agents), solubilizing agents, preservative agents, buffer agents, and tensioactive agents, among others. Such agents are provided in amounts that are not substantially toxic to retinal pigment epithelial cells when administered to an eye.

In one embodiment, the excipient component of the present compositions comprises a cyclodextrin component present in an amount that is less toxic to retinal pigment epithelial cells relative to an equal amount of an excipient selected from the group consisting of polysorbate 80 and benzyl alcohol. The excipient component comprises substantially no polysorbate 80 or benzyl alcohol, such as less than 0.05% (w/v) of benzyl alcohol.

The cyclodextrin component can comprise one or more cyclodextrins or cyclodextrin derivatives. In a specific embodiment, the cyclodextrin component comprises at least one cyclodextrin selected from the group consisting of sulfobutyl ether 4-beta-cyclodextrin, hydroxypropyl beta-cyclodextrin, and hydroxypropyl gamma-cyclodextrin. In one embodiment, the cyclodextrin component is present in an amount from about 0.5% (w/v) to about 5.0% (w/v) of the composition. In other embodiments, the cyclodextrin component may be present in an amount from about 0.1% (w/v) to about 10% (w/v), for example, 0.1% (w/v) to about 5% (w/v).

In another embodiment, a method of treating an ocular condition of an individual person or animal comprises administering the present compositions to the interior of an eye of the individual, such as the vitreous or posterior segment of the eye.

The present invention also provides methods of screening potential ophthalmic excipients for toxicity, such as RPE cell toxicity. The present methods provide for the ability to determine the toxicity of a potential excipient based on standardized values and/or in relation to other excipients in use. Such methods generally comprise a step of contacting cultured retinal pigment epithelial cells with an excipient. The cell viability and/or morphology can be determined. By exposing cultured RPE cells to different concentrations of an excipient or combinations of excipients, it is possible to evaluate the toxicity of such excipients and determine potentially useful amounts of such excipients for use in the present drug delivery systems.

Each and every feature described herein, and each and every combination of two or more of such features, is included within the scope of the present invention provided that the features included in such a combination are not mutually inconsistent. In addition, any feature or combination of features may be specifically excluded from any embodiment of the present invention.

Additional aspects and advantages of the present invention are set forth in the following description, drawings, and claims, particularly when considered in conjunction with the accompanying examples.

### BRIEF DESCRIPTION OF THE DRAWINGS

FIG. 1 is a graph illustrating cell viability (%) as a function of carboxymethyl cellulose (CMC) concentration.

FIG. 2 is a graph and photographs illustrating cell morphology score as a function of CMC concentration at 24 hour, 48 hour, and 72 hour time points.

FIG. 3 is a graph illustrating cell viability (%) as a function of hydroxypropylmethyl cellulose (HPMC) concentration.

FIG. 4 is a graph and photographs illustrating cell morphology score as a function of HPMC concentration at 24 hour, 48 hour, and 72 hour time points.

FIG. 5 is a graph illustrating cell viability (%) as a function of poloxamer 407 nf (poloxamer) concentration.

FIG. 6 is a graph and photographs illustrating cell morphology score as a function of poloxamer concentration at 24 hour, 48 hour, and 72 hour time points.

FIG. 7 is a graph illustrating cell viability (%) as a function of hyaluronic acid (HA) concentration.

FIG. 8 is a graph and photographs illustrating cell morphology score as a function of HA concentration at 24 hour, 48 hour, and 72 hour time points.

FIG. 9 is a graph illustrating cell viability (%) as a function of hydroxypropyl gamma-cyclodextrin (hydroxypropyl gamma-CD) concentration.

FIG. 10 is a graph and photographs illustrating cell morphology score as a function of hydroxypropyl gamma-CD concentration at 24 hour, 48 hour, and 72 hour time points.

FIG. 11 is a graph illustrating cell viability (%) as a function of sulfobutyl ether 4 beta-cyclodextrin (sulfobuytyl ether 4 beta-CD) concentration.

FIG. 12 is a graph and photographs illustrating cell morphology score as a function of sulfobutyl ether 4 beta-CD concentration at 24 hour, 48 hour, and 72 hour time points.

FIG. 13 is a graph illustrating cell viability (%) as a function of hydroxypropyl beta-cyclodextrin (hydroxypropyl beta-CD) concentration.

FIG. 14 is a graph and photographs illustrating cell morphology score as a function of hydroxypropyl beta-CD concentration at 24 hour, 48 hour, and 72 hour time points.

FIG. 15 is a graph and photographs illustrating cell viability (%) as a function of benzyl alcohol (benzylOH) concentration.

FIG. 16 is a graph and photographs illustrating cell morphology score as a function of benzylOH concentration at 24 hour, 48 hour, and 72 hour time points.

FIG. 17 is a graph illustrating cell viability (%) as a function of borate buffer (X Eur. Ph. Borate Buffer) concentration.

FIG. 18 is a graph and photographs illustrating cell morphology score as a function of borate buffer concentration at 24 hour, 48 hour, and 72 hour time points.

FIG. 19 is a graph illustrating cell viability (%) as a function of phosphate buffer (X phosphate) concentration.

FIG. 20 is a graph illustrating cell morphology score as a function of phosphate buffer concentration at 24 hour, 48 hour, and 72 hour time points.

FIG. 21 is a graph illustrating cell viability (%) as a function of polysorbate 80 concentration.

FIG. 22 is a graph and photographs illustrating cell morphology score as a function of polysorbate 80 concentration at 24 hour, 48 hour, and 72 hour time points.

FIG. 23 is a series of photographs illustrating cell morphology characteristics used in scoring the RPE cell cultures.

### DESCRIPTION

Compositions and methods have been invented which provide effective treatment of ocular conditions, such as disorders or diseases of the posterior segment of an eye of an individual, such as a human or animal. The present compositions comprise a therapeutic component and an excipient component. The excipient component preferably includes one or more agents that are not substantially toxic to retinal cells, including retinal epithelial cells. Thus, the present compositions have a reduced toxicity relative to existing intraocular compositions used for treating ocular conditions, such as Kenalog®-40. The present therapeutic ophthalmic compositions and methods are effective in alleviating or reducing one or more symptoms associated with ocular conditions. Thus, the present invention relates to new compositions and methods employing retinal friendly excipients or excipients that do not damage, injure, or otherwise significantly harm retinal cells of the individual being administered the compositions.

The present therapeutic ophthalmic compositions comprise a therapeutic component and an excipient component. The therapeutic component is present in an amount effective in providing a desired therapeutic effect to an individual, such as a human or animal patient, when the composition is administered to the interior of an eye of the individual. Thus, it may be understood that the present compositions are useful for injection into the interior of an eye of the individual. More specifically, the present compositions are useful for injection or other administration into the posterior segment of the eye.

The therapeutic component of the present compositions comprises one or more therapeutic agents, such as chemical compounds, macromolecules, proteins, and the like, which are effective in treating an ocular condition, such as an ocular condition of the posterior segment of an eye. In certain embodiments, the therapeutic agents are poorly soluble in the composition. For example, the therapeutic agents may be present as particles in the composition.

Therapeutic agents which may be provided in the therapeutic component of the present ophthalmic compositions may be obtained from public sources or may be synthesized using routine chemical procedures known to persons of ordinary skill in the art. Agents are screened for therapeutic efficacy using conventional assays known to persons of ordinary skill in the art. For example, agents can be monitored for their effects on reducing intraocular pressure, reducing or preventing neovascularization in the eye, reducing inflammation in the eye, and the like using such conventional assays. Thus, the therapeutic component of the present systems can comprise a variety of therapeutic agents, including anti-angiogenesis agents, anti-inflammatory agents, neuroprotective agents, and the like

For example, the therapeutic component of the present compositions may comprise one or more of the following: anti-excitotoxic agents, anti-histamine agents, anti-biotic agents, beta blocker agents, one or more steroid agents, anti-neoplastic agents, immunosuppressive agents, anti-viral agents, anti-oxidant agents, anti-inflammatory agents, adrenergic receptor agonists and antagonists, and neuroprotective agents.

Examples of antihistamines include, and are not limited to, loradatine, hydroxyzine, diphenhydramine, chlorpheniramine, brompheniramine, cyproheptadine, terfenadine, clemastine, triprolidine, carbinoxamine, diphenylpyraline, phenindamine, azatadine, tripelennamine, dexchlorpheniramine, dexbrompheniramine, methdilazine, and trimprazine doxylamine, pheniramine, pyrilamine, chiorcyclizine, thonzylamine, and derivatives thereof.

As used herein, the term “derivative” refers to any substance which is sufficiently structurally similar to the material of which it is identified as a derivative so as to have substantially similar functionality or activity, for example, therapeutic effectiveness, as the material when the substance is used in place of the material. Useful derivatives of a substance can be routinely determined or identified by conducting one or more conventional assays using the derivatives instead of the substance from which the derivative is derived.

Examples of antibiotics include without limitation, cefazolin, cephradine, cefaclor, cephapirin, ceftizoxime, cefoperazone, cefotetan, cefutoxime, cefotaxime, cefadroxil, ceftazidime, cephalexin, cephalothin, cefamandole, cefoxitin, cefonicid, ceforanide, ceftriaxone, cefadroxil, cephradine, cefuroxime, cyclosporine, ampicillin, amoxicillin, cyclacillin, ampicillin, penicillin G, penicillin V potassium, piperacillin, oxacillin, bacampicillin, cloxacillin, ticarcillin, azlocillin, carbenicillin, methicillin, nafcillin, erythromycin, tetracycline, doxycycline, minocycline, aztreonam, chloramphenicol, ciprofloxacin hydrochloride, clindamycin, metronidazole, gentamicin, lincomycin, tobramycin, vancomycin, polymyxin B sulfate, colistimethate, colistin, azithromycin, augmentin, sulfamethoxazole, trimethoprim, gatifloxacin, ofloxacin, and derivatives thereof.

Examples of beta blockers include acebutolol, atenolol, labetalol, metoprolol, propranolol, timolol, and derivatives thereof.

Examples of steroids include corticosteroids, such as cortisone, prednisolone, flurometholone, dexamethasone, medrysone, loteprednol, fluazacort, hydrocortisone, prednisone, betamethasone, prednisone, methylprednisolone, triamcinolone hexacatonide, paramethasone acetate, diflorasone, fluocinonide, fluocinolone, triamcinolone, triamcinolone acetonide, derivatives thereof, and mixtures thereof.

Examples of antineoplastic agents include adriamycin, cyclophosphamide, actinomycin, bleomycin, duanorubicin, doxorubicin, epirubicin, mitomycin, methotrexate, fluorouracil, carboplatin, carmustine (BCNU), methyl-CCNU, cisplatin, etoposide, interferons, camptothecin and derivatives thereof, phenesterine, taxol and derivatives thereof, taxotere and derivatives thereof, vinblastine, vincristine, tamoxifen, etoposide, piposulfan, cyclophosphamide, and flutamide, and derivatives thereof.

Examples of immunosuppresive agents include cyclosporine, azathioprine, tacrolimus, and derivatives thereof.

Examples of antiviral agents include interferon gamma, zidovudine, amantadine hydrochloride, ribavirin, acyclovir, valciclovir, dideoxycytidine, phosphonoformic acid, ganciclovir and derivatives thereof.

Examples of antioxidant agents include ascorbate, alpha-tocopherol, mannitol, reduced glutathione, various carotenoids, cysteine, uric acid, taurine, tyrosine, superoxide dismutase, lutein, zeaxanthin, cryotpxanthin, astazanthin, lycopene, N-acetyl-cysteine, carnosine, gamma-glutamylcysteine, quercitin, lactoferrin, dihydrolipoic acid, citrate, Ginkgo Biloba extract, tea catechins, bilberry extract, vitamins E or esters of vitamin E, retinyl palmitate, and derivatives thereof.

Other therapeutic agents include squalamine, carbonic anhydrase inhibitors, brimonidine, prostamides, prostaglandins, antiparasitics, antifungals, tyrosine kinase inhibitors, glutamate receptor antagonists, including NMDA receptor antagonists, and derivatives thereof.

In view of the foregoing, it can be appreciated that therapeutic component of the present compositions can comprise many different types of therapeutic agents, and that such agents are routinely known to persons of ordinary skill in the art.

The excipient component of the present compositions comprises one or more retinal friendly excipient agents or otherwise inert substances. Retinal friendly excipient agents contribute to the enhanced compatibility and tolerance of the present compositions to the tissues in the posterior segment of the eye, for example, the retina of the eye, relative to compositions previously proposed for intravitreal injection into a posterior segment of an eye, for example, the composition sold under the trademark Kenalog®-40. Examples of excipients which may be present in the compositions include retinal friendly or retinal compatible solubilizing agents, surfactant or tensioactive agents, preservative agents, viscosity inducing or viscosing agents, tonicity agents, and the like.

Viscosing agents include, without limitation, sodium carboxymethylcellulose (CMC), hydroxypropylmethyl cellulose (HPMC), poloxamer 407 nf (Pluronic® F127 Prill), and hyaluronic acid.

Solubilizing agents include without limitation, cyclodextrins (CDs), such as hydroxypropyl gamma-CD (Cavasol®), sulfobutyl ether 4 beta-CD (Captisol®), and hydroxypropyl beta-CD (Kleptose®). Polysorbate 80 (Tween80®) may also be understood to be a solubilizer or resuspension agent.

Preservative agents may include benzyl alcohol, as well as others, as discussed herein.

Buffer agents may include phosphate buffers, such as dibasic sodium phosphate heptahydrate, monobasic sodium phosphate monohydrate; and/or borate buffers, such as sodium borate, boric acid, sodium chloride (according to Eu. Pharmacopeia).

Tensioactive agents may include NaCl or sugar alcohols such as manitol.

The present compositions comprise an excipient component which comprises one or more excipients or excipient agents. The excipient component is provided in an amount that is less toxic to retinal pigment epithelial cells than an equal amount of benzyl alcohol or polysorbate 80. Thus, the compositions may be understood to comprise excipients that are less toxic than excipients currently used in ophthalmic compositions. Administration of the compositions to the interior of the eye advantageously provide reduced inflammation compared to existing ophthalmic compositions.

Certain embodiments of the present compositions comprise a cyclodextrin component associated with a therapeutic component to improve or enhance the therapeutic efficacy and/or bioavailability of the therapeutic component. For example, the cyclodextrin component may be associated with the therapeutic component to enhance the solubility of the therapeutic component in the composition, enhance or improve the stability of the therapeutic component in the composition or in the eye, and/or enhance or improve the ocular tolerability of the composition and/or therapeutic component, relative to compositions which comprise the same therapeutic component and substantially no cyclodextrin component.

In certain embodiments, the excipient component of the present compositions comprises a cyclodextrin component, such as one or more types of different cyclodextrins or cyclodextrin derivatives. For example, the cyclodextrin component may comprise a cyclodextrin selected from the group consisting of alpha-cyclodextrin, beta-cyclodextrin, gamma-cyclodextrin, derivatives thereof, and mixtures thereof. The term “cyclodextrin derivative” has the broadest meaning generally understood in the art, and refers to a compound or a mixture of compounds wherein one or more of the free hydroxyl groups of alpha-, beta-, or gamma-cyclodextrin is replaced with any other group. A “water-soluble” cyclodextrin derivative is soluble at a concentration of at least 300 mg/mL in water. The cyclodextrin derivative used in the compositions disclosed herein may vary. Derivatives of alpha-cyclodextrin, beta-cyclodextrin, and gamma-cyclodextrin may be used. In certain compositions, a beta-cyclodextrin derivative such as calcium sulfobutylether-beta-cyclodextrin, sodium sulfobutylether-beta-cyclodextrin, and hydroxypropyl-beta-cyclodextrin, may be used. Alternatively, a gamma-cyclodextrin derivative such as calcium sulfobutylether-gamma-cyclodextrin, sodium sulfobutylether-gamma-cyclodextrin, and hydroxypropyl-gamma-cyclodextrin may be used. Some specific derivatives contemplated herein are the hydroxypropyl derivatives of cyclodextrins, such as hydroxypropyl-beta-cyclodextrin or hydroxypropyl-gamma-cyclodextrin.

The present excipient components also comprise substantially no polysorbate 80 or benzyl alcohol. As discussed herein, polysorbate 80 and/or benzyl alcohol are believed to be responsible for retinal pigment epithelial cell toxicity associated with existing intraocular ophthalmic formulations. Thus, embodiments of the present compositions comprise an excipient component that comprises a cyclodextrin component and substantially no polysorbate 80 or benzyl alcohol. In other words, and in certain embodiments, the present compositions are substantially free of added preservative components, or include effective preservative components which are more compatible with or friendly to the posterior segment, e.g., retina or RPE, of the eye relative to certain concentrations of benzyl alcohol, which is included in the Kenalog®-40 composition as a preservative. In addition, the present compositions may include no added resuspension component or a resuspension component which is more compatible with or friendly to the posterior segment, e.g., retina, of the eye relative to polysorbate-80, which is included in the Kenalog®-40 composition. The cyclodextrin component is present in these embodiments of the composition in an amount that is less toxic to retinal pigment epithelial cells relative to an equal amount of either polysorbate 80 or benzyl alcohol.

The present compositions are preferably in an injectable form. In other words, the compositions may be intraocularly administered, such as by intravitreal injection, using a syringe and needle or other similar device (e.g., see U.S. patent Publication No. 2003/0060763). The present compositions are preferably in a liquid form. For example, the present compositions may comprise a liquid carrier which contains the therapeutic component and the excipient component. The liquid carrier may be an aqueous material, such as water or saline. Thus, the present compositions may comprise an ophthalmically acceptable aqueous based vehicle component suitable for administration to the interior of the eye. The present compositions may be understood to be intraocular formulations, such as suspensions, solutions, emulsions (such as oil-containing emulsions, such as oil-in-water emulsions or water-in-oil emulsions), and the like.

The present compositions are injectable into the interior of an eye of an individual without causing significant adverse effects related to the presence of the compositions. For example, the present compositions preferably do not cause substantial unwanted changes in intraocular pressure of the eye resulting from the injection of the composition into the eye. While not wishing to be bound by any particular theory or mechanism of action, embodiments of the present compositions may have a greater viscosity compared to topically applied ophthalmic compositions. The greater viscosity may be helpful in maintaining a desired intraocular pressure compared to topical compositions. Thus, embodiments of the present compositions may have a viscosity that is greater than a topical ophthalmic composition that comprises the same therapeutic component and cyclodextrin component.

In addition, the present intraocularly administerable compositions can comprise greater concentrations of the therapeutic component relative to topical ophthalmic compositions. However, by administering the compositions directly into the eye, smaller volumes of the compositions can be employed to obtain a desired therapeutic effect. By administering the present compositions directly into the eye, such as the vitreous of the eye, relatively more of the therapeutic component of the composition can be present in the eye to provide a desired therapeutic effect. As discussed herein, the amount of therapeutic component provided in topical ophthalmic compositions must be relatively greater since the composition gets washed by tears and drains into nasolacrimal ducts eventhough the composition may comprise a lower concentration of the therapeutic component. Similarly, the cyclodextrin component of the excipient component can be provided in greater amounts relative to cyclodextrins provided in topical ophthalmic compositions.

As discussed herein, the therapeutic component comprises one or more therapeutic agents. In certain embodiments of the present compositions, the therapeutic component comprises, consists essentially of, or consists of, steroids and/or steroid precursors. As used herein, a steroid precursor is understood to be an agent that can be converted into a therapeutically effective steroid by physiological processes. Steroid precursors may be understood to be steroid prodrugs. An example of a steroid precursor or steroid prodrug is a compound that is converted in vivo into a steroid after the compound is administered into the eye. For example, a prednisolone precursor is a compound that is converted to prednisolone in vivo. A dexamethasone precursor is a compound that is converted to dexamethasone in vivo. A triamcinolone precursor is a compound that is converted to triamcinolone in vivo. Steroids and steroid precursors can be obtained from commercial suppliers, or can be synthesized using routine methods known to persons of ordinary skill in the art, and can be screened using conventional methods known to persons of ordinary skill in the art. The steroid or steroid precursor may be present in the compositions as a plurality of particles.

As discussed herein, the therapeutic component may comprise one or more therapeutic agents that are poorly soluble. For example, the therapeutic agent may have a limited solubility in water, for example, at 25 degrees C. In certain embodiments, the therapeutic component comprises a therapeutic agent that has a solubility in water at 25 degrees C. of less than 10 mg/ml. The therapeutic component should be ophthalmically acceptable, that is, should have substantially no significant or undue detrimental effect of the eye structures or tissues. Embodiments comprising a corticosteroid component have an ability of such component to reduce inflammation in the posterior segment of the eye into which the composition is placed caused by the result of one or more diseases and/or conditions in the posterior segment of the eye.

In at least one embodiment of the present compositions, the therapeutic component comprises, consists essentially of, or consists entirely at least one steroid selected from the group consisting of cortisone, dexamethasone, fluorometholone, loteprednol, medrysone, prednisolone, prednisolone acetate, triamcinolone, and triamcinolone acetonide.

Corticosteroids may be present in an amount of at least about 10 mg per ml of the composition. One important advantage of the present invention is the effective ability of the present compositions to include relatively large amounts or concentrations of the corticosteroids, or other therapeutic agents.

Thus, the therapeutic component may be present in the present compositions in an amount in the range of about 1% or less to about 5% or about 10% or about 20% or about 30% or more (w/v) of the composition. Providing relatively high concentrations or amounts of the therapeutic component in the present compositions is beneficial in that reduced amounts of the composition may be required to be placed or injected into the posterior segment of the eye in order to provide the same amount or more of the therapeutic component in the posterior segment of the eye relative to compositions, such as Kenalog®-40, which include less than 4% (w/v) of the corticosteroid. Thus, in one very useful embodiment, the present compositions include more than about 4% (w/v), for example at least about 5% (w/v), to about 10% (w/v) or about 20% (w/v) or about 30% (w/v) of the corticosteroid component.

In some embodiments of the present compositions, the cyclodextrin component is provided in an amount from about 0.1% (w/v) to about 5% (w/v) of the composition. In further embodiments, the cyclodextrin comprises up to about 10% (w/v) of certain cyclodextrins, as discussed herein. The excipient component of the present compositions may comprise one or more types of cyclodextrins or cyclodextrin derivatives, such as alpha-cyclodextrins, beta-cyclodextrins, gamma-cyclodextrins, and derivatives thereof. As understood by persons of ordinary skill in the art, cyclodextrin derivatives refer to any substituted or otherwise modified compound that has the characteristic chemical structure of a cyclodextrin sufficiently to function as a cyclodextrin, for example, to enhance the solubility and/or stability of therapeutic agents and/or reduce unwanted side effects of the therapeutic agents and/or to form inclusive complexes with the therapeutic agents. In certain embodiments, the cyclodextrin component comprises at least one cyclodextrin selected from the group consisting of sulfobutyl ether 4-beta-cyclodextrin, hydroxypropyl beta-cyclodextrin, and hydroxypropyl gamma-cyclodextrin.

As discussed herein, embodiments of the present compositions comprise a cyclodextrin component present in an amount that has a reduced toxicity to retinal pigment epithelial cells relative to an equal amount of polysorbate 80 or benzyl alcohol. In certain embodiments of the present compositions, the cyclodextrin component comprises an amount of hydroxypropyl gamma-cyclodextrin from about 0.1% (w/v) to about 10% (w/v) of the composition. Certain embodiments may comprise an amount of sulfobutyl ether 4-beta-cyclodextrin from about 0.1% (w/v) to about 10% (w/v). Further embodiments may comprise an amount of hydroxypropyl beta-cyclodextrin from about 0.1% (w/v) to about 5% (w/v).

The present compositions may also comprise one or more other excipients in addition to those described above. For example, the present implants may include effective amounts of buffering agents, preservatives and the like, which have a reduced toxicity, such as a reduced toxicity relative to polysorbate 80 or benzyl alcohol.

Certain compositions may include a viscosing component or a viscosity inducing component, such as a polymer component that is effective in stabilizing the therapeutic component in the composition. The viscosity inducing component is present in an effective amount in increasing, advantageously substantially increasing, the viscosity of the composition. Without wishing to limit the invention to any particular theory of operation, it is believed that increasing the viscosity of the compositions to values well in excess of the viscosity of water, for example, at least about 100 cps at a shear rate of 0.1/second, compositions which are highly effective for placement, e.g., injection, into the posterior segment of an eye of a human or animal are obtained. Along with the advantageous placement or injectability of the present compositions into the posterior segment, the relatively high viscosity of the present compositions are believed to enhance the ability of the present compositions to maintain the therapeutic component, including therapeutic component particles, in substantially uniform suspension in the compositions for prolonged periods of time, for example, for at least about one week, without requiring resuspension processing. The relatively high viscosity of the present compositions may also have an additional benefit of at least assisting the compositions to have the ability to have an increased amount or concentration of the therapeutic component, as discussed elsewhere herein, for example, while maintaining such therapeutic component in substantially uniform suspension for prolonged periods of time.

Embodiments of the present compositions have viscosities of at least about 10 cps or at least about 100 cps or at least about 1000 cps, more preferably at least about 10,000 cps and still more preferably at least about 70,000 cps or more, for example up to about 200,000 cps or about 250,000 cps or more, at a shear rate of 0.1/second. The present compositions not only have the relatively high viscosity as noted above but also have the ability or are structured or made up so as to be effectively placeable, e.g., injectable, into a posterior segment of an eye of a human or animal, preferably through a 27 gauge needle, or even through a 30 gauge needle.

The presently useful viscosity inducing components preferably are shear thinning components in that as the present composition containing such a shear thinning viscosity inducing component is passed or injected into the posterior segment of an eye, for example, through a narrow space, such as 27 gauge needle, under high shear conditions the viscosity of the composition is substantially reduced during such passage. After such passage, the composition regains substantially its pre-injection viscosity so as to maintain the therapeutic component in suspension in the eye.

Any suitable viscosity inducing component, for example, ophthalmically acceptable viscosity inducing component, may be employed in the present compositions. Many such viscosity inducing components have been proposed and/or used in ophthalmic compositions used on or in the eye. The viscosity inducing component is present in an amount effective in providing the desired viscosity to the composition. Advantageously, the viscosity inducing component is present in an amount in a range of about 0.5% or about 1.0% to about 5% or about 10% or about 20% (w/v) of the composition. The specific amount of the viscosity inducing component employed depends upon a number of factors including, for example and without limitation, the specific viscosity inducing component being employed, the molecular weight of the viscosity inducing component being employed, the viscosity desired for the present composition being produced and/or used and the like factors. The viscosity inducing component is chosen to provide at least one advantage, and preferably multiple advantages, to the present compositions, for example, in terms of each of injectability into the posterior segment of the eye, viscosity, sustainability of the therapeutic component in suspension, for example, in substantially uniform suspension, for a prolonged period of time without resuspension processing, compatibility with the tissues in the posterior segment of the eye into which the composition is to be placed and the like advantages. More preferably, the selected viscosity inducing component is effective to provide two or more of the above-noted benefits, and still more preferably to provide all of the above-noted benefits.

The viscosity inducing component preferably comprises a polymeric component and/or at least one viscoelastic agent, such as those materials which are useful in ophthalmic surgical procedures. Examples of useful viscosity inducing components include, but are not limited to, hyaluronic acid, carbomers, polyacrylic acid, cellulosic derivatives, polycarbophil, polyvinylpyrrolidone, gelatin, dextrin, polysaccharides, polyacrylamide, polyvinyl alcohol, polyvinyl acetate, derivatives thereof and mixtures thereof.

The molecular weight of the presently useful viscosity inducing components may be in a range of about 10,000 Daltons or less to about 2 million Daltons or more. In one particularly useful embodiment, the molecular weight of the viscosity inducing component is in a range of about 100,000 Daltons or about 200,000 Daltons to about 1 million Daltons or about 1.5 million Daltons. Again, the molecular weight of the viscosity inducing component useful in accordance with the present invention, may vary over a substantial range based on the type of viscosity inducing component employed, and the desired final viscosity of the present composition in question, as well as, possibly one or more other factors.

In one very useful embodiment, a viscosity inducing component is a hyaluronate component, for example, a metal hyaluronate component, preferably selected from alkali metal hyaluronates, alkaline earth metal hyaluronates and mixtures thereof, and still more preferably selected from sodium hyaluronates, and mixtures thereof. The molecular weight of such hyaluronate component preferably is in a range of about 50,000 Daltons or about 100,000 Daltons to about 1.3 million Daltons or about 2 million Daltons. In one embodiment, the present compositions include a hyaluronate component in an amount in a range about 0.05% to about 0.5% (w/v). In a further useful embodiment, the hyaluronate component is present in an amount in a range of about 1% to about 4% (w/v) of the composition. In this latter case, the very high polymer viscosity forms a gel that slows particle sedimentation rate to the extent that often no resuspension processing is necessary over the estimated shelf life, for example, at least about 2 years, of the composition. Such a composition may be marketed in pre-filled syringes since the gel cannot be easily removed by a needle and syringe from a bulk container. In one embodiment, the polymer component comprises hyaluronic acid.

The excipient component of the present compositions may comprise additional agents in addition to the cyclodextrin component. However, the excipient component remains substantially free of polysorbate 80 or benzyl alcohol. For example, the excipient component preferably contains no polysorbate 80 or benzyl alcohol, but may contain some trace amounts so long as such amounts are not toxic to ocular tissue, such as retinal pigment epithelium. For example, benzyl alcohol may be provided in an amount less than 0.05% (w/v). In certain embodiments, the excipient component may further comprise an excipient selected from the group consisting of carboxymethyl cellulose, hydroxypropylmethyl cellulose, boric acid, and salts thereof. As discussed herein, the excipients are preferably retinal friendly. Such excipients can be obtained from public sources, or produced using conventional methods known to persons of ordinary skill in the art. Excipients can be screened for toxicity to retinal cells using cytotoxicity assays known to persons of ordinary skill in the art, and as described herein.

The present compositions may also include at least one buffer component in an amount effective to control the pH of the composition and/or at least one tonicity component in an amount effective to control the tonicity or osmolality of the compositions. More preferably, the present compositions include both a buffer component and a tonicity component, which may include one or more sugar alcohols, such as manitol, or salts, such as sodium chloride, as discussed herein. The buffer component and tonicity component may be chosen from those which are conventional and well known in the ophthalmic art. Examples of such buffer components include, but are not limited to, acetate buffers, citrate buffers, phosphate buffers, borate buffers and the like and mixtures thereof. Phosphate buffers are particularly useful. Useful tonicity components include, but are not limited to, salts, particularly sodium chloride, potassium chloride, any other suitable ophthalmically acceptably tonicity component and mixtures thereof.

The amount of buffer component employed preferably is sufficient to maintain the pH of the composition in a range of about 6 to about 8, more preferably about 7 to about 7.5. The amount of tonicity component employed preferably is sufficient to provide an osmolality to the present compositions in a range of about 200 to about 400, more preferably about 250 to about 350, mOsmol/kg respectively. Advantageously, the present compositions are substantially isotonic.

In view of the disclosure herein, it can be understood that the excipient component may comprise one or more excipient agents provided in amounts from about 0.1% to about 10% (w/v) of the composition. Examples of specific amounts of agents include 0.5% of a cyclodextrin, 0.5% of a vitamin E agent, 2% hyaluronic acid, 2% of a vitamin E agent, and 5% of a cyclodextrin. The exact amounts can be determined by measuring the toxicity of such excipient agents in vitro, as described herein, or by administering formulations or drug delivery systems with desired amounts into the interior of the eye and monitoring the effects of such exposure to retinal cells or the eye or individual in general.

For example, an in vivo method that may be useful to determine the desired amount of excipients to provide in the present compositions may comprise administering an injectable composition into an eye of the animal. Different compositions comprising different amounts and/or combinations of excipients may be administered to eyes of different animals. The animals and eyes can be monitored and/or examined for viability, clinical effects, and gross ocular effects. In certain methods, the effects can be monitored by slit lamp biomicroscopy, pupillary reflex, ophthalmoscopy, electroretinography (ERG), intraocular pressure (IOP), body weight, macroscopic observations, and microscopic pathololgy of ocular tissues. Dose response curves can be obtained based on the results of such methods, and the desired amounts of the excipient agents can be determined. Results which indicate that compositions having a certain amount of an excipient do not produce inflammation, irritation, or other adverse side effects compared to control compositions may be indicative that such excipient-containing compositions have a low retinal cell toxicity.

The present compositions may include one or more other components in amounts effective to provide one or more useful properties and/or benefits to the present compositions. For example, although the present compositions may be substantially free of added preservative components, in other embodiments, the present compositions include effective amounts of preservative components, preferably such components which are more compatible with or friendly to the tissue in the posterior segment of the eye into which the composition is placed than benzyl alcohol. Examples of such preservative components include, without limitation, benzalkonium chloride, methyl and ethyl parabens, hexetidine, chlorite components, such as stabilized chlorine dioxide, metal chlorites and the like, other ophthalmically acceptable preservatives and the like and mixtures thereof. The concentration of the preservative component, if any, in the present compositions is a concentration effective to preserve the composition, and is often in a range of about 0.00001% to about 0.05% or about 0.1% (w/v) of the composition.

In addition, embodiments of the present composition may include an effective amount of resuspension component effective to facilitate the suspension or resuspension of the therapeutic component in the present compositions. As noted above, in certain embodiments, the present compositions are free of added resuspension components. In other embodiments of the present compositions effective amounts of resuspension components are employed, for example, to provide an added degree of insurance that the therapeutic component remains in suspension, as desired and/or can be relatively easily resuspended in the present compositions, such resuspension be desired. Advantageously, the resuspension component employed in accordance with the present invention, if any, is chosen to be more compatible with or friendly to the tissue in the posterior segment of the eye into which the composition is placed than polysorbate 80. In other words, the resuspension component has a reduced toxicity to posterior segment ocular tissues compared to polysorbate 80.

Any suitable resuspension component may be employed in accordance with the present invention. Examples of such resuspension components include, without limitation, surfactants such as poloxanes, for example, sold under the trademark Pluronic®; tyloxapol; sarcosinates; polyethoxylated castor oils, other surfactants and the like and mixtures thereof. One very useful class of resuspension components are those selected from vitamin derivatives. Although such materials have been previously suggested for use as surfactants in ophthalmic compositions, they have been found to be effective in the present compositions as resuspension components. Examples of useful vitamin derivatives include, without limitation, Vitamin E tocopheryl polyethylene glycol succinates, such as Vitamin E tocopheryl polyethylene glycol 1000 succinate (Vitamin E TPGS). Other useful vitamin derivatives include, again without limitation, Vitamin E tocopheryl polyethylene glycol succinamides, such as Vitamin E tocopheryl polyethylene glycol 1000 succinamide (Vitamin E TPGS) wherein the ester bond between polyethylene glycol and succinic acid is replaced by an amide group.

The presently useful resuspension components are present, if at all, in the compositions in accordance with the present invention in an amount effective to facilitate suspending the therapeutic agent, such as therapeutic agent particles, in the present compositions, for example, during manufacture of the compositions or thereafter. The specific amount of resuspension component employed may vary over a wide range depending, for example, on the specific resuspension component being employed, the specific composition in which the resuspension component is being employed and the like factors. Suitable concentrations of the resuspension component, if any, in the present compositions are often in a range of about 0.01% to about 5%, for example, about 0.02% or about 0.05% to about 1.0% (w/v) of the composition. However, as discussed herein, the resuspension components should be present in amounts with little toxicity to retinal pigment epithelial cells.

In one embodiment of the present compositions, an effective amount of a solubilizing component is provided in the composition to solubilize a minor amount, that is less than 50%, for example in a range of 1% or about 5% to about 10% or about 20% of a corticosteroid component. For example, the inclusion of a cyclodextrin component, such as beta-cyclodextrin, secondary butylether beta-cyclodextrin, other cyclodextrins and the like and mixtures thereof, at about 0.5 to about 5.0% (w/v) solubilizes about 1 to about 10% of the initial dose of triamcinolone acetonide. This presolubilized fraction provides a readily bioavailable loading dose, thereby avoiding any delay time in therapeutic effectiveness. The use of such a solubilizing component may provide a relatively quick release of the corticosteroid component into the eye for therapeutic effectiveness. Such solubilizing component, of course, should be ophthalmically acceptable or at least sufficiently compatible with the posterior segment of the eye into which the composition is placed to avoid undue damage to the tissue in such posterior segment.

In view of the disclosure herein, one useful embodiment of the present compositions comprises a therapeutic component present in an amount effective in providing a desired therapeutic effect to an individual when the composition is administered to the interior of an eye of the individual; and at least one cyclodextrin selected from the group consisting of sulfobutyl ether 4-beta-cyclodextrin, hydroxypropyl beta-cyclodextrin, and hydroxypropyl gamma-cyclodextrin. As discussed herein, the composition is substantially free of polysorbate 80 or benzyl alcohol.

In another embodiment of the present compositions, a therapeutic ophthalmic composition useful for injection into a posterior segment of an eye of an individual, comprises a therapeutic component present in an amount effective in providing a desired therapeutic effect to an individual when the composition is administered to the interior of an eye of the individual; and a cyclodextrin component present in an amount from about 0.5% (w/v) to about 5.0% of the composition and effective in solubilizing a therapeutic agent of the therapeutic component. Such an amount of the cyclodextin component may be effective in solubilizing about 50% or less of the therapeutic agent of the therapeutic component. In accordance with the disclosure herein, such an amount of a cyclodextrin component, at least in certain embodiments, provides a reduced toxicity relative to an equal amount of polysorbate 80 or benzyl alcohol.

By utilizing amounts of the cyclodextrin component or other excipients which have a reduced toxicity relative to equal amounts of polysorbate 80 or benzyl alcohol, the present ophthalmic compositions may be understood to have a reduced toxicity relative to a second substantially identical composition which comprises polysorbate 80 or benzyl alcohol, or both, and which may be substantially free of a cyclodextrin component.

In addition, other specific embodiments of the present drug compositions may comprise one or more excipients selected from the group consisting of polysorbate 80, benzyl alcohol, poloxamer 407 nf, sodium carboxymethylcellulose, hydroxypropylmethyl cellulose, and hyaluronic acid provided that such excipients are present in amounts that have a low toxicity. For example, such compositions comprise an excipient component in an amount that does not substantially affect cell viability or cell morphology, or both. For example, the effects mediated by such excipients results in a reduction in cell viability and cell morphology less than 50% compared to systems without such excipients.

The toxicity of potentially useful ophthalmic excipients can be determined by contacting cultured RPE cells with an excipient. Some detailed procedures are described in the Examples herein. Broadly, a method of screening excipients in accordance with the present invention may comprise a step of contacting cultured RPE cells with an excipient. Generally, the method can be practiced by contacting cultured RPE cells with different concentrations of an excipient at one or more time points. The cultured cells may be examined to determine the effects, such as toxicity, of the excipients on the cells. For example, the viability of the cells may be examined by evaluating the metabolism of the cells, such as by using a colorometric assay. In addition, and/or alternatively, the morphology of the cells may be examined by scoring the cell cultures based on visual critieria, such as cell size and shape.

Suitable methods for screening excipients may include culturing RPE cells (such as ARPE-19 cells) in culture dishes and conducting a dose-response for excipients at different time points, such as 24 hours, 48 hours, and 72 hours. Various properties of excipient-containing incubating solutions, such as pH, osmolarity (mOsm), and viscosity, can be measured. Concentrations of the excipients can be determined using routine methods, and can include concentrations commonly used in ophthalmic formulations, concentrations with desired solubility characteristics, and/or limiting the concentrations with desirable viscosity, osmolarity, and/or pH values. The methods may also comprise one or more steps of measuring cell proliferation, secretion of pro-inflammatory mediators, and the like.

In addition, as discussed herein, the present screening methods may comprise a step of placing an excipient containing composition in an animal's eye. Dose response curves can be obtained using these in vivo screening procedures. From the dose response curve data, the desired amounts can be determined for the present compositions.

The present compositions can be produced using conventional techniques routinely known by persons of ordinary skill in the art. For example, a therapeutic component and an excipient component can be combined in dry form or in a liquid carrier. The composition can be sterilized. In certain embodiments, such as preservative-free embodiments, the compositions can be sterilized and packaged in single-dose amounts. The compositions may be prepackaged in intraocular dispensers which can be disposed of after a single administration of the unit dose of the compositions.

The present compositions can be prepared using suitable blending/processing techniques, for example, one or more conventional blending techniques. The preparation processing should be chosen to provide the present compositions in forms which are useful for placement or injection into the posterior segments of eyes of humans or animals. In one useful embodiment a concentrated therapeutic component dispersion is made by combining the therapeutic component with water, and the excipients (other than the viscosity inducing component) to be included in the final composition. The ingredients are mixed to disperse the therapeutic component and then autoclaved. The viscosity inducing component may be purchased sterile or sterilized by conventional processing, for example, by filtering a dilute solution followed by lyophylization to yield a sterile powder. The sterile viscosity inducing component is combined with water to make an aqueous concentrate. The concentrated therapeutic component dispersion is mixed and added as a slurry to the viscosity inducing component concentrate. Water is added in a quantity sufficient (q.s.) to provide the desired composition and the composition is mixed until homogenous.

In certain embodiments, the cyclodextrin component and therapeutic component are present as complexes in the composition or when administered to the interior of an eye. Complexation of the cyclodextrin component and a therapeutic agent of the therapeutic component can occur via routine methods known to persons of ordinary skill in the art. For example, complexation of a cyclodextrin component and a therapeutic agent can be accomplished by ultrasonic processing with a high energy microtip sonicator at ambient temperatures. Such a process is effective for processing small volumes of solution. Larger volumes can be processed by autoclaving the mixture at elevated temperatures, such as about 120 degrees C. Excess uncomplexed therapeutic agent can be removed by centrifugation and filtration. Or, as another example, inclusion complexes can be made by:(i) rapid stirring at 25 degrees C. for 72 hrs, (ii) high-shear processing at 60 degrees C. with a rotor/stator homogenizer, (iii) brief ultrasonication with a high-energy probe sonicator, and (iv) autoclaving in sealed borosilicate glass vials for 10 min at 121 degrees C. Equimolar concentration of therapeutic agent, such as a steroid, can be added to 10% solutions of cyclodextrin in dilute (20 mM) aqueous buffer prior to complex formation. After processing, aliquots are filtered (0.45 μm) for HPLC analysis of soluble, complexed therapeutic agent and the hydrolytic degradant, non-esterified therapeutic agent. For example, see U.S. patent Publication No. 2002/0198174 (Lyons).

In one embodiment, a sterile, viscous, suspension suitable for injection is made using a corticosteroid, such as triamcinolone. A process for producing such a composition may comprise two main stages, namely, sterile suspension bulk compounding and asceptic filling. The bulk product manufacturing can include steps of producing three separate parts or components followed by asceptic combination of these three parts. The asceptic filling operation can be conducted in a class 100 environment, and the sterile bulk product can be filled into pre-sterilized ready-to-use syringes.

One method of manufacturing a steril bulk suspension is described below.

Part I of the bulk product is prepared in a main batch vessel that has capabilities of bulk heat sterilization and viscous fluid mixing. First, water for injection (WFI) at 40% of batch size is charged into the vessel and sodium chloride is dissolved. Triamcinolone powder is then added and dispersed with strong agitation. The suspension is heated and sterilized at above 121° C. for a sufficient time period by steam passing through the jacket of the vessel. After the bulk heat cycle is completed, the suspension is cooled down to room temperature.

Part II of the bulk product is prepared in an open vessel equipped with a top entering, variable speed mixer. First, WFI at 10% of batch size is charged into the vessel. Sodium phosphate salts and, optionally, a beta-cyclodextrin derivative is added and dissolved. If necessary, the pH of the solution is adjusted with 1 N sodium hydroxide and/or 1 N hydrochloric acid.

Part III of the bulk product is prepared in a Class 100 environment through a series of aseptic procedures. First, sodium hyaluronate is dissolved in WFI at a dilute concentration. The solution is sterile filtered and sodium hyaluronate powder is recovered through lyophilization. Finally, the sodium hyaluronate powder is reconstituted with sterile WFI at 50% of batch size.

The sterile bulk suspension is compounded by aseptically combining the three parts described above. First, the Part II solution is filtered into a sterile Part I composition in the main batch vessel using a 0.2 micron sterilizing grade filter. Part III is then aseptically transferred into the main batch vessel. Finally, the bulk is mixed to achieve uniformity. The final bulk suspension is held in a controlled area before aseptic filling.

Aseptic filling is performed in a Class 100 environment. The sterile bulk suspension is first filtered through a clarification screen into a sterile holding container. The bulk suspension is then transferred to the filling machine and filled into pre sterilized syringes. The filled syringes or units are transferred to the packaging area for application of tamper-evident seals, labeling and cartoning.

The foregoing method can be used to produce compositions, such as a sterile injectable gel suspension comprising 2% (w/w) triamcinolone acetonide, 2.5% (w/w) sodium hyaluronate, 0.63% (w/w) sodium chloride, 0.3% (w/w) dibasic sodium phosphate, heptahydrate, 0.04% (w/w) monobasic sodium phosphate, monohydrate, and water for injection. The method can also be used to produce a sterile injectable gel suspension comprising 8% (w/w) triamcinolone acetonide, 2.3% (w/w) sodium hyaluronate, 0.63% sodium chloride, 0.3% dibasic sodium phosphate, heptahydrate, 0.04% (w/w) monobasic sodium phosphate, monohydrate, and water for injection. Typically, in such compositions, the concentration of sodium hyaluronate is from about 2% (w/w) to about 3% (w/w).

Methods of using the present compositions are provided and are included within the scope of the present invention. In general, such methods comprise administering a composition in accordance with the present invention to a posterior segment of an eye of a human or animal, thereby obtaining a desired therapeutic effect. The administering step advantageously comprises at least one of intravitreal injecting, subconjunctival injecting, sub-tenon injecting, retrobulbar injecting, suprachoroidal injecting and the like. A syringe apparatus including an appropriately sized needle, for example, a 27 gauge needle or a 30 gauge needle, can be effectively used to inject the composition with the posterior segment of an eye of a human or animal.

Among the diseases/conditions which can be treated or addressed in accordance with the present invention include, without limitation, the following:

MACULOPATHIES/RETINAL DEGENERATION: Non-Exudative Age Related Macular Degeneration (ARMD), Exudative Age Related Macular Degeneration (ARM D), Choroidal Neovascularization, Diabetic Retinopathy, Acute Macular Neuroretinopathy, Central Serous Chorioretinopathy, Cystoid Macular Edema, Diabetic Macular Edema.

UVEITIS/RETINITIS/CHOROIDITIS: Acute Multifocal Placoid Pigment Epitheliopathy, Behcet's Disease, Birdshot Retinochoroidopathy, Infectious (Syphilis, Lyme, Tuberculosis, Toxoplasmosis), Intermediate Uveitis (Pars Planitis), Multifocal Choroiditis, Multiple Evanescent White Dot Syndrome (MEWDS), Ocular Sarcoidosis, Posterior Scleritis, Serpignous Choroiditis, Subretinal Fibrosis and Uveitis Syndrome, Vogt-Koyanagi-Harada Syndrome.

VASCULAR DISEASES/EXUDATIVE DISEASES: Retinal Arterial Occlusive Disease, Central Retinal Vein Occlusion, Disseminated Intravascular Coagulopathy, Branch Retinal Vein Occlusion, Hypertensive Fundus Changes, Ocular Ischemic Syndrome, Retinal Arterial Microaneurysms, Coat's Disease, Parafoveal Telangiectasis, Hemi-Retinal Vein Occlusion, Papillophlebitis, Central Retinal Artery Occlusion, Branch Retinal Artery Occlusion, Carotid Artery Disease (CAD), Frosted Branch Angitis, Sickle Cell Retinopathy and other Hemoglobinopathies, Angioid Streaks, Familial Exudative Vitreoretinopathy, Eales Disease.

TRAUMATIC/SURGICAL: Sympathetic Ophthalmia, Uveitic Retinal Disease, Retinal Detachment, Trauma, Laser, PDT, Photocoagulation, Hypoperfusion During Surgery, Radiation Retinopathy, Bone Marrow Transplant Retinopathy.

PROLIFERATIVE DISORDERS: Proliferative Vitreal Retinopathy and Epiretinal Membranes, Proliferative Diabetic Retinopathy.

INFECTIOUS DISORDERS: Ocular Histoplasmosis, Ocular Toxocariasis, Presumed Ocular Histoplasmosis Syndrome (POHS), Endophthalmitis, Toxoplasmosis, Retinal Diseases Associated with HIV Infection, Choroidal Disease Associated with HIV Infection, Uveitic Disease Associated with HIV Infection, Viral Retinitis, Acute Retinal Necrosis, Progressive Outer Retinal Necrosis, Fungal Retinal Diseases, Ocular Syphilis, Ocular Tuberculosis, Diffuse Unilateral Subacute Neuroretinitis, Myiasis.

GENETIC DISORDERS: Retinitis Pigmentosa, Systemic Disorders with Accosiated Retinal Dystrophies, Congenital Stationary Night Blindness, Cone Dystrophies, Stargardt's Disease and Fundus Flavimaculatus, Best's Disease, Pattern Dystrophy of the Retinal Pigmented Epithelium, X-Linked Retinoschisis, Sorsby's Fundus Dystrophy, Benign Concentric Maculopathy, Bietti's Crystalline Dystrophy, pseudoxanthoma elasticum.

RETINAL TEARS/HOLES: Retinal Detachment, Macular Hole, Giant Retinal Tear.

TUMORS: Retinal Disease Associated with Tumors, Congenital Hypertrophy of the RPE, Posterior Uveal Melanoma, Choroidal Hemangioma, Choroidal Osteoma, Choroidal Metastasis, Combined Hamartoma of the Retina and Retinal Pigmented Epithelium, Retinoblastoma, Vasoproliferative Tumors of the Ocular Fundus, Retinal Astrocytoma, Intraocular Lymphoid Tumors.

MISCELLANEOUS: Punctate Inner Choroidopathy, Acute Posterior Multifocal Placoid Pigment Epitheliopathy, Myopic Retinal Degeneration, Acute Retinal Pigement Epithelitis and the like.

The present methods may comprise a single injection into the posterior segment of an eye or may involve repeated injections, for example over periods of time ranging from about one week or about 1 month or about 3 months to about 6 months or about 1 year or longer.

Thus, the present compositions can be administered to an individual, such as a person or animal, to treat one or more ocular conditions. Thus, the present invention relates to methods of treating a posterior segment ocular condition or conditions.

### EXAMPLES

The following non-limiting examples provide those of ordinary skill in the art with specific preferred methods to treat conditions within the scope of the present invention and are not intended to limit the scope of the invention.

### Example 1

### Cytotoxicity of Excipient Agents on Retinal Pigment Epithelial Cells

Cell viability and cell morphology were examined to evaluate the toxicity of excipients on retinal pigment epithelial cells. The human retina cell line used in these experiments is the ARPE-19 cell line (human adult-derived retinal pigmented epithelial cells). The ARPE-19 cell line is non-transformed and displays physiological characteristics close to freshly isolated RPE from donor (Dunn, K C et al., (1996) “ARPE-19, a human retinal pigment epithelial cell line with differentiated properties”, Exp. Eye Res, 62:155-69). These cells form stable monolayers, which exhibit morphological and functional polarity. The cells exhibit morphological polarization when plated on laminin-coated filters in medium with a low serum concentration (Dunn K C et al., supra). They form tight-junctions with transepithelial resistance of monolayers (Dunn K C et al., supra). From a molecular standpoint, it appears that ARPE-19 express a huge pattern of genes similar to those expressed by human RPE from fresh explant which could account for their physiological function (Klimanskaya I. et al., “Derivation and comparative assessment of retinal pigment epithelium from human embryonic stem cells using transcriptomics”, Cloning and Stem Cells, 2004, 6(3):217-45). ARPE-19 cells express RPE-specific markers such as cellular retinaldehyde-binding protein (CRALBP) (Crabb J W et al., “Cloning of the cDNAs encoding the cellular retinaldehyde-binding protein from bovine and human retina and comparison of the protein structures”, J Biol Chem., 1988, 263(35):18688-92) and RPE-65 protein (Hamel C P et al., “Molecular cloning and expression of RPE65, a novel retinal pigment epithelium-specific microsomal protein that is post-transcriptionally regulated in vitro,” J Biol Chem., 1993, 268(21)15751-7. Comparison of ARPE-19 cells to the human transformed RPE cell line D407 shows that the latter is unable to maintain a intense polarity like ARPE-19 cells exhibit (Rogojina A T et al., “Comparing the use of Affymetrix to spotted oligonucleotide microarrays using two retinal pigment epithelium cell lines”, Molecular Vision, 2003, 9:482-96). Also, ARPE-19 cells are described to possess phagocytosis activity when differentiated.

ARPE-19 cells are widely used as a retinal model that resemble physiological properties of RPE cells. Similar methods of culturing ARPE-19 cells and cytotoxicity assays can be found in Yeung et al., “Cytotoxicity of triamcinolone on cultured human retinal pigment epithelial cells: comparison with dexamethasone and hydrocortisone”, Jpn J. Ophthalmol, 2004; 48:236-242. Cell viability and cell morphology were examined using conventional colormetric and visual methods. Viability and morphology measurements were obtained at 24 hours, 48 hours, and 72 hours after exposure to an excipient composition.

To assess cell viability, mitochondrial metabolism was quantified through conventional colorometric assays (i.e., the MTT assay). This calorimetric assay utilizes 3-(4,5-dimethylthiazol-2-yl)-2,5-diphenyltetrazolium bromide (MTT) and correlates mitochondrial metabolism and cell viability through measurement of dehydrogenase activity which converts a substrate into crystal. More specifically, the assay measures the activity of living cells through mitochondrial dehydrogenases. When dissolved in culture cell medium, MTT solution appears dark orange. Mitochondrial dehydrogenases of viable cells degrade MTT by cleaving the tetrazolium ring yielding to formation of a purple formazan crystal, which is insoluble in water. Crystals are subsequently dissolved in isopropanol solution. A resulting purple solution is spectrophotometrically measured. Cell viability is relative to amount of formazan and calculated as a percentage of remaining treated compared to non-treated cells.

Results from the MTT assay were expressed as a percentage of cell viability calculated using the following equation:
% cell viability=ODtest/ODcontrol×100.

For each experiment, one concentration was performed in triplicate. Each point was spectrophotometrically read twice. Average of readings was calculated, then average from these 3 values was determined.

After 3 experiments were independently completed under the same conditions, a graph was plotted with cell viability expressed as a function of concentration (dose response) and for various incubation period (time course). The IC₅₀ was then estimated based on the graphed data.

More specifically, cell density in 24-multiwell plates was observed on the day of the experiment to check if confluence of the cells was reached. Aliquots of MTT concentrated solutions were removed from a freezer at −20° C. and thawed at room temperature. Cell medium was warmed at 37° C. before use. Appropriated tubes for diluting compounds were prepared. Higher concentrations of each excipient agent was prepared. Serial dilution was then performed in cell medium (DMEM:F12+10% FBS).

Cell culture medium in the 24-well plate was removed using vacuum-pump. Cells were then stimulated with 0.5 ml final volume for each concentration of one given excipient agent. Distribution of the excipient-containing solution was performed using automated pipet-aid in sterile environment (under PSM) or not depending on time of compounds and duration of incubation.

After stimulation, cells were replaced in a CO₂ incubator for the required time of exposure. All remaining solutions were kept in small containers (stored in lab tank) before being disposed (according to current method describing proper use of dangerous substance).

The MTT solution was made by reconstituting MTT powder in PBS 1× at a concentration of 5 mg/ml, then aliquoted by 12 ml and stored at −20° C. until use. The MTT solution was prepared in culture medium supplemented with FBS to a final concentration of 0.5 mg/ml. The solution was kept at 37° C. before adding to cells.

A stock solubilizing solution was prepared and stored at 4° C. for 6 months. It was composed of 10% TritonX-100, 10% HCl 1N in 80% isopropanol. Before use, the solution was brought to room temperature.

After incubating time, the cells were removed from the incubator to stop the reaction. Medium was discarded for predefined experiment or removed using vacuum-pump. 0.5 ml MTT solution was added per well in 24-well plate. Cells were then incubated at 37° C., 10% CO2 for 3 h. MTT was converted into formazan crystals. Cells were removed from incubator and 0.5 ml of solubilizing solution was added. A blank sample was prepared by adding MTT and solubilizing solution to 1:1 ratio. Plates were placed on rotative shaker to around 300 rpm for 1 h to gently mix and enhance dissolution of crystals. If necessary, the solution was pipetted to help dissolution in case of dense cultures.

Samples were analyzed by spectrophotometry using MRXII predefined program. To this end, 200 μl from each point of 24-well plates were loaded in duplicate in 96-multiwell plates. Absorbance was measured at 570 nm and was compared to 690 nm background from plastic of multiwell microplates. Results were automatically printed and raw data archived.

After appropriate number of experiments are completed in the same conditions, a graph is plotted expressing % cells viability as a function of both concentrations and time of exposure. When the graph profile allowed for IC₅₀ determination, concentration range is approximately deduced from curve.

Care was taken to store the reconstituted MTT solution under conditions which reduce decomposition and erroneous results. In addition, care was taken to reduce microbial contamination, and maintining desirable protein concentrations.

Cell morphology was visualized using light microscopy. Cell morphology observation using light microscopy permits determination of (i) cell number and density and (ii) whether or not cells in contact with an excipient agent display modified phenotype compared to a non-treated population of cells.

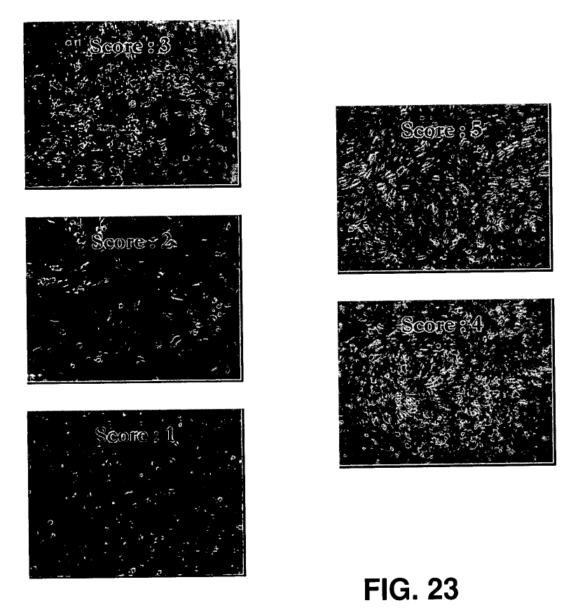

Cell morphology was analyzed in parallel by semi-quantitative scoring ranging from 5 to 1, from basal to lethal phenotype respectively, as shown in FIG. 23.

More specifically, cells were removed from an incubator for examination. Morphological shape of cells was visualized using a light microscope and CCD camera. Every 3 wells of a given concentration was observed through image analysis software. Then, a representative photograph describing the average appearance of cells was saved. The supernatant was either, kept for further predefined experiments to perform, or removed using vacuum-pump.

Morphology semi-quantitative scoring was obtained using the following typical phenotype scale, as shown in FIG. 23. Resulting semi-quantitative scoring is represented as a function of time course and dose response. Score 5 : wild type phenotype of non-treated cells, 100% confluent adherent cells. This phenotype very much vary following post-seeding time. For example, at confluence ARPE-19 cells at 1 day appear well defined with visible outer membrane and dark grey cytoplasm. After 3 days of confluence, limits between cells become less visible and cells adopt a more hexagonal shape and constitute an epithelium-like uniform dense structure. Score 4: density <100%. Cell shape has changed, some spaces are present between cells, few cells possibly detached. Score 3: 80% <Cell density <40%. Spaces are sometimes present among cells, areas appears confluent while in others, cells detached. General cell shape starts showing non negligible alterations. Score 2: Cell density <50%. Cells adopt exclusively stressed appearance, presence of mass dead floating cells. Score 1: Cell density <10%. Presence of almost exclusively dead cells.

Based on quotation, each condition of the applied compound was given a score which allow to a morphology evolution profile to be prepared, ie. cell morphology as a function of compound concentration and time of exposure.

MTT and morphological quotation results were globally interpreted so to discriminate between excipient agents that do not modify cell parameters from those that affect only one of them or from those affecting both.

By determining both cell viability and cell morphology for treated cells, the effects of excipient agents can be quantified in a reproducible manner. The analysis of compound-induced effects on ARPE-19 cells according these two end-points enables one to discriminate between an agent without any noticeable effect on cells, and agents which irritate cells through morphological modification but without affecting metabolism, irritate cells by affecting metabolism without morphological modification, or irritate cells by affecting both cell shape and viability.

Cell morphology was examined upon treatment with increasing concentrations and times of exposure to excipient agents. In addition, cell viability was measured based on mitochondrial enzyme activity. The physico-chemical properties of excipient-containing incubating solutions such as pH, osmolarity were also determined.

ARPE-19 cells (ATCC CRL-2302) were purchased from LGC Promochem (Molsheim, France). Culture dishes were obtained from BD Falcon (le Pont de Claix, France). DMEM:F12 1:1 mixture, foetal bovine serum (FBS: USDA approved), penicillin/streptomycin (10000 unit 10000 μg) were purchased from Cambrex (Verviers, Belgium). Trypsin/EDTA was purchased from InVitrogen (Cergy Pontoise, France). ProlineXL dispenser was obtained from Biohit (Bonnelles, France). H₂O₂, isopropanol, Triton X-100 and MTT lyophilized powder were obtained from from Sigma-Aldrich (St Quentin Fallavier, France).

The MRXII microplate reader (Dynex Technologies) was purchased from ThermoLifeSciences (Cergy Pontoise, France), Z1 Coulter counter was obtained from Beckman Coulter (Villepinte, France). The orbital shaker (Heidolph Instruments) was obtained from Fisher Bioblock (IllKirch, France).

The excipients: Cavasol® (Wacker; batch # 83B009), Captisol® (Cydex; batch # CDDR-059-46), Kleptose® (RM #R14080), HA (Hyaluron Inc.; batch # 04-001), HPMC (Methocel F4M premium RM #1018), CMC (type 7H3SXF 10-15 RM #1392), Pluronic®F127 Prill (RM #1230), boric acid (PM #12550) and sodium borate (PM #1980) were provided by Allergan (Irvine, Calif.). Benzyl alcohol (RM #11006), Tween80® (RM #1044), sodium chloride (PM #1979), sodium phosphate monobasic monohydrate (PM #1095) and disodium hydrogen phosphate heptahydrate (PM #1116) were purchased from Sigma-Aldrich (St Quentin Fallavier, France).

Borate buffer solution according to European Pharmacopeia was prepared with 2.5 g NaCl, 2.85 g disodium tetraborate and 10.5 g boric acid dissolved in 1000 ml of water. Therefore the concentration referenced as X for Borate buffer is 42 mM NaCl, 7.5 mM disodium tetraborate and 170 mM boric acid. A 3× concentrated borate buffer solution was prepared in water. Then, various concentrations were obtained by dilution in culture medium for each condition. Concentration of borate buffer solution according to European Pharmacopeia is indicated as X. Dilutions applied on experiments are : 0.12×, 0.15×, 0.2×, 0.25×, 0.5×, 0.75×.

Phosphate buffer is composed of dibasic and monobasic phosphate at constant proportion ratio. The resulting concentration of phosphate buffer containing both entities is referenced as X. Phosphate buffer was used in triamcinolone acetonide formulations at X=(0.3% w/v dibasic phosphate−0.04% w/v monobasic phosphate). Concentrations inferior (0.16×, 0.33×) and superior (1.6×, 3.3×, 5×, 6.6×) to X have been tested in addition to X in order to determine IC₅₀ concentration range. To this end, 20 ml of 6.7×(the most concentrated solution) is prepared by weighing 0.4 g of dibasic phosphate and 0.053 g of monobasic phosphate added in culture medium. Then, subsequent conditions are obtained through serial dilutions in culture medium supplemented with 10% FBS (fetal bovine serum).

Higher concentrated solutions for each excipient were obtained by weighing appropriate amounts or pipet adequate volumes of stock powder or solution and diluting in culture media DMEM:F12 supplemented with 10% FBS. Then, subsequent concentrations were obtained by serial dilution of concentrated solution into the same media.

ARPE-19 cells (passage 9 to 27) were seeded the day prior to experimentation in 24 well-plates at 125,000 cells/well in DMEM:F12 medium supplemented with 10% FBS. Time courses and dose responses were simultaneously performed on ARPE-19 cells. Parameters of incubating solutions were measured such as pH, osmolarity for every concentration of each compound. Viscosity was also determined when applicable. Times of incubation are 24 h, 48 h, 72 h. Negative (non-treated) and positive controls (5 mM H₂O₂) were included at each time point. Not-treated condition was cell culture medium supplemented with serum. 5 mM H₂O₂ was prepared from 3% H₂O₂ stock solution (875 mM).

Generally, a first experiment which covers a wide range of concentrations was performed. If preliminary results (see Experiment 1 of Table 1) showed conditions are appropriate to determine IC₅₀, a second set of experiments was performed to confirm previous data. If not, the concentration range was modified to determine more accurately compound concentrations leading to inhibition of 50% of cell viability (see Experiments 2, 3, and 4 of Table 1).

Concentrations of excipient agents applied to cells were determined considering several parameters, such as commonly used concentration in formulations, limiting concentration to excipient agent solubility, limiting concentration to applicable osmolarity and pH values. All ranges of concentrations for each excipient agent were obtained with serial dilution from most concentrated condition into cell culture medium (DMEM:F12 supplemented with 10% FBS).

TABLE 1
Range of tested concentrations.
Number of Experiment

| | 1 | 2 | 3 | 4 |
|---|---|---|---|---|
| Polysorbate 80 | 0.1˜20% | 0.01˜0.1% | 0.01˜0.1% | 0.01˜0.1% |
| Benzyl alcohol | 0.05˜2% | 0.05˜2% | 0.05˜2% | 0.05˜2% |
| Borate buffer | 0.12˜0.75X⁽¹⁾ | not performed | not performed | not performed |
| Phosphate buffer | 0.16˜6.6X⁽²⁾ | 0.16˜6.6X⁽²⁾ | 0.16˜6.6X⁽²⁾ | 0.16˜6.6X⁽²⁾ |
| Poloxamer 407nf | 0.1˜10% | 0.05˜5% | 0.05˜5% | 0.05˜5% |
| Sodium carboxymethyl | 0.2˜1.2% | 0.2˜1.2% | 0.2˜1.2% | 0.2˜1.2% |
| cellulose | | | | |
| Hydroxypropyl | 0.2˜1.2% | 0.2˜1.2% | 0.2˜1.2% | 0.2˜1.2% |
| methyl cellulose | | | | |
| Hyaluronic acid | 0.2˜1.2% | 0.2˜1.2% | 0.2˜1.2% | 0.2˜1.2% |
| Hydroxypropyl gamma- | 0.1˜20% | 0.05˜10% | 0.05˜10% | 0.05˜10% |
| CD | | | | |
| Sulfobutyl ether 4 beta- | 0.1˜20% | 0.05˜10% | 0.05˜10% | 0.05˜10% |
| CD | | | | |
| Hydroxypropyl beta-CD | 0.1˜20% | 0.01˜5% | 0.01˜5% | 0.01˜5% |

⁽¹⁾X: Concentration of borate buffer from European Pharmacopeia
⁽²⁾X: Concentration of phosphate buffer in formulations X = (0.3% (w/v) dibasic phosphate - 0.04% (w/v) monobasic phosphate)

Parameters of incubating solutions such as pH, osmolarity were measured for every concentration of each excipient agent. pH was measured using pHM220 MeterLab (Villeurbanne, France) connected to InLab 427 electrode from Mettler Toledo (Urdorf, Switzerland). Osmolarity was measured using osmometer type 13/13DR from Roebling (Berlin, GR).

The pH of the incubating solutions for the cell cultures was maintained at about 7.6 (range 7.5 to 7.7). The osmolarity of the incubating solutions was maintained within a range from about 300 mOsm to about 700 mOsm (range from 307 mOsm to 710 mOsm). The osmolarity varied as a function of the concentration of the excipient in the incubation solution.

For each concentration of tested compound, pH and osmolarity parameters were measured. If values are far from physiological conditions (pH ≈7.4 and osmolarity ≈300 mOsm), the corresponding concentration was not further tested although preliminary results display remarkable results so to maintain the condition.

TABLE 2
pH and osmolarity of excipients dilutions

| | | | | | | | |
|---|---|---|---|---|---|---|---|
| Poloxamer | 0 | 0.05% | 0.1% | 0.5% | 1% | 5% | 10% |
| 407nf | | | | | | | |
| pH | 7.6 | 7.5 | 7.5 | 7.5 | 7.5 | 7.7 | 7.7 |
| mOsm | 309 | 309 | 307 | 325 | 331 | 346 | 462 |
| CMC | 0 | 0.2% | 0.4% | 0.6% | 0.8% | 1.0% | 1.0% |
| pH | 7.5 | 8 | 8.1 | 8.1 | 8.1 | 8.2 | 8.2 |
| mOsm | 305 | 306 | 312 | 314 | 329 | 344 | 344 |
| HPMC | 0 | 0.2% | 0.4% | 0.6% | 0.8% | 1.0% | 1.2% |
| pH | 7.5 | 8 | 8 | 8 | 8 | 8 | 8 |
| mOsm | 305 | 302 | 308 | 307 | 310 | 341 | 327 |
| HA | 0 | 0.2% | 0.4% | 0.6% | 0.8% | 1% | 1.2% |
| pH | 7.5 | 8.1 | 8.1 | 8.1 | 8.0 | 7.9 | 8.0 |
| mOsm | 305 | 305 | 311 | 314 | 319 | 344 | 342 |
| Hydroxypropyl | 0 | 0.1% | 0.5% | 1% | 5% | 10% | 20% |
| gamma-CD | | | | | | | |
| pH | 7.6 | 7.6 | 7.6 | 7.6 | 7.6 | 7.6 | 7.6 |
| mOsm | 309 | 308 | 317 | 325 | 384 | 481 | 689 |
| Sulfobutylether | 0 | 0.1% | 0.5% | 1% | 5% | 10% | 20% |
| 4beta-CD | | | | | | | |
| pH | 7.6 | 7.6 | 7.6 | 7.6 | 7.6 | 7.7 | 7.7 |
| mOsm | 309 | 310 | 318 | 333 | 408 | 518 | 710 |
| Hydroxypropyl | 0 | 0.1% | 0.5% | 1% | 5% | 10% | 20% |
| beta-CD | | | | | | | |
| pH | 7.6 | 7.6 | 7.6 | 7.5 | 7.5 | 7.5 | 7.6 |
| mOsm | 309 | 309 | 317 | 325 | 375 | 459 | 635 |
| Benzyl | 0 | 0.05% | 0.1% | 0.5% | 1% | 1.5% | 2% |
| alcohol | | | | | | | |
| pH | 7.5 | 8 | 8.1 | 8 | 7.9 | 7.9 | 8 |
| mOsm | 305 | 293 | 307 | 347 | 398 | 444 | 493 |
| Borate | 0 | 0.12X | 0.15X | 0.2X | 0.25X | 0.5X | 0.75X |
| buffer | | | | | | | |
| pH | 7.9 | 7.9 | 7.9 | 7.9 | 7.9 | 7.9 | 7.7 |
| mOsm | 300 | 323 | 323 | 330 | 335 | 366 | 416 |
| Phosphate | 0 | 0.16X | 0.33X | 1.6X | 3.3X | 5X | 6.6X |
| buffer | | | | | | | |
| pH | 7.5 | 8 | 8 | 7.8 | 7.7 | 7.6 | 7.7 |
| mOsm | 305 | 300 | 308 | 339 | 386 | 427 | 478 |
| Polysorbate 80 | 0 | 0.01% | 0.02% | 0.04% | 0.06% | 0.08% | 0.1% |
| pH | 7.5 | 7.5 | 7.6 | 7.5 | 7.5 | 7.5 | 7.6 |
| mOsm | 309 | 310 | 307 | 313 | 346 | 390 | 523 |

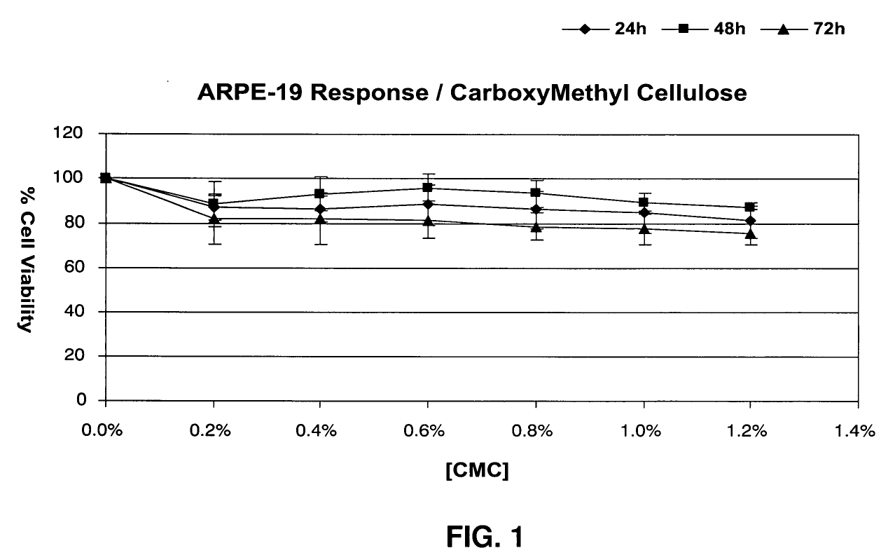

These data allow one to conclude for example that Sulfobutylether 4 beta-CD at 20% far exceeds physiological osmolarity range, leading one not to consider this condition further. In comparison, 10% hydroxypropyl gamma-CD solution also displayed high osmolarity values but gave unexpected results using both assay characterizations (see FIGS. 1 & 2). This means this condition is maintained in the tested concentration range so to determine IC₅₀ value.

### Example 2

### Sodium Carboxymethylcellulose (CMC)

The cytotoxicity of CMC (low density) on ARPE-19 cells upon various conditions of concentrations and exposure period was studied. Concentration ranges from 0.2 to 1.2% was applied to cells. Then the MTT assay was performed and results are shown in FIG. 1. The general profile shows that CMC appears of low cytotoxicity. Addition of 0.2% dose seems to weakly decrease cell viability, but raising content of CMC (up to 1.2%) does not further diminish the number of viable cells. FIG. 1 also shows that longer duration of exposure does not supplementary impact cell capacity to transform MTT into crystals.

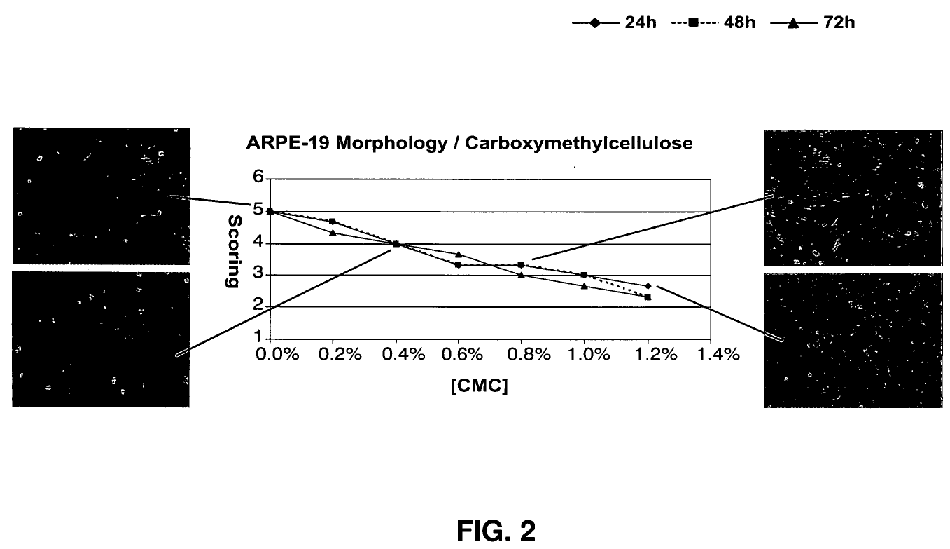

Cell morphology using light microscopy was analyzed (FIG. 2). From the score results, the addition of CMC is not without consequence on cell shape. At 0.2%, a slight modification is noticeable at all time points. Increasing the concentration of CMC augments the impact on cells. The highest tested concentration (i.e., 1.2%) results in a stressed phenotype (see photographs of FIG. 2) although cells still form a monolayer. 0.8% to 1% appear to be the first concentrations bringing alterations to cell shape. With regards to viability results, it can be concluded that although cells seem affected in their aspect, it does not correlate with striking metabolic changes leading to cell death.

This CMC-induced cytotoxicity could be owed to physical constraints due to osmolarity or pH properties of incubating solutions (see Table 1) or also its viscosity (data not shown). Although physico-chemical constraints do not seem to alter cell metabolism (no IC₅₀ range concentrations are reached), it certainly influences morphological aspect of ARPE-19 cells.

The raw data for cell viability and cell morphology are provided below

Cell Viability

24 Hours

| [CMC] (%) | 1 | 2 | 3 | Mean (%) | SD × 100 |
|---|---|---|---|---|---|
| 0 | 100.0 | 100.0 | 100.0 | 100.0 | 0 |
| 0.2 | 85.5 | 92.4 | 82.6 | 86.8 | 5.0 |
| 0.4 | 85.7 | 92.8 | 81.2 | 86.5 | 5.8 |
| 0.6 | 90.3 | 96.2 | 78.3 | 88.3 | 9.1 |
| 0.8 | 88.9 | 92.9 | 76.5 | 86.1 | 8.5 |
| 1.0 | 86.9 | 92.5 | 74.7 | 84.7 | 9.1 |
| 1.2 | 89.9 | 74.0 | 81.0 | 81.6 | 8.0 |

48 Hours

| [CMC] (%) | 1 | 2 | 3 | Mean (%) | SD × 100 |
|---|---|---|---|---|---|
| 0 | 100.0 | 100.0 | 100.0 | 100.0 | 0 |
| 0.2 | 77.1 | 92.8 | 95.3 | 88.4 | 9.9 |
| 0.4 | 84.9 | 94.7 | 99.5 | 93.0 | 7.4 |
| 0.6 | 87.6 | 97.7 | 101.0 | 95.4 | 7.0 |
| 0.8 | 86.8 | 93.7 | 99.2 | 93.2 | 6.2 |
| 1.0 | 83.9 | 90.0 | 93.2 | 89.0 | 4.7 |
| 1.2 | 86.7 | 88.2 | 87.4 | 87.5 | 0.7 |

72 Hours

| [CMC] (%) | 1 | 2 | 3 | Mean (%) | SD × 100 |
|---|---|---|---|---|---|
| 0 | 100.0 | 100.0 | 100.0 | 100.0 | 0 |
| 0.2 | 69.3 | 86.2 | 89.8 | 81.8 | 11.0 |
| 0.4 | 69.1 | 90.2 | 87.5 | 82.3 | 11.5 |
| 0.6 | 72.8 | 88.9 | 83.1 | 81.6 | 8.1 |
| 0.8 | 76.8 | 85.6 | 73.9 | 78.7 | 6.1 |
| 1.0 | 80.2 | 84.5 | 69.8 | 78.2 | 7.6 |
| 1.2 | 74.6 | 80.7 | 71.7 | 75.7 | 4.6 |

Cell Morphology

24 Hours

| [CMC] (%) | 1 | 2 | 3 | Mean |
|---|---|---|---|---|
| 0 | 5 | 5 | 5 | 5.0 |
| 0.2 | 5 | 5 | 4 | 4.7 |
| 0.4 | 4 | 4 | 4 | 4.0 |
| 0.6 | 4 | 3 | 3 | 3.3 |
| 0.8 | 4 | 3 | 3 | 3.3 |
| 1.0 | 3 | 3 | 3 | 3.0 |
| 1.2 | 3 | 2 | 3 | 2.7 |

48 Hours

| [CMC] (%) | 1 | 2 | 3 | Mean |
|---|---|---|---|---|
| 0 | 5 | 5 | 5 | 5.0 |
| 0.2 | 4 | 5 | 5 | 4.7 |
| 0.4 | 4 | 4 | 4 | 4.0 |
| 0.6 | 4 | 3 | 3 | 3.3 |
| 0.8 | 4 | 3 | 3 | 3.3 |
| 1.0 | 3 | 3 | 3 | 3.0 |
| 1.2 | 3 | 2 | 2 | 2.3 |

72 Hours

| [CMC] (%) | 1 | 2 | 3 | Mean |
|---|---|---|---|---|
| 0 | 5 | 5 | 5 | 5.0 |
| 0.2 | 4 | 5 | 4 | 4.3 |
| 0.4 | 4 | 4 | 4 | 4.0 |
| 0.6 | 4 | 4 | 3 | 3.7 |
| 0.8 | 3 | 3 | 3 | 3.0 |
| 1.0 | 3 | 2 | 3 | 2.7 |
| 1.2 | 3 | 2 | 2 | 2.3 |

### Example 3

### HydroxypropvImethyl Cellulose (HPMC)

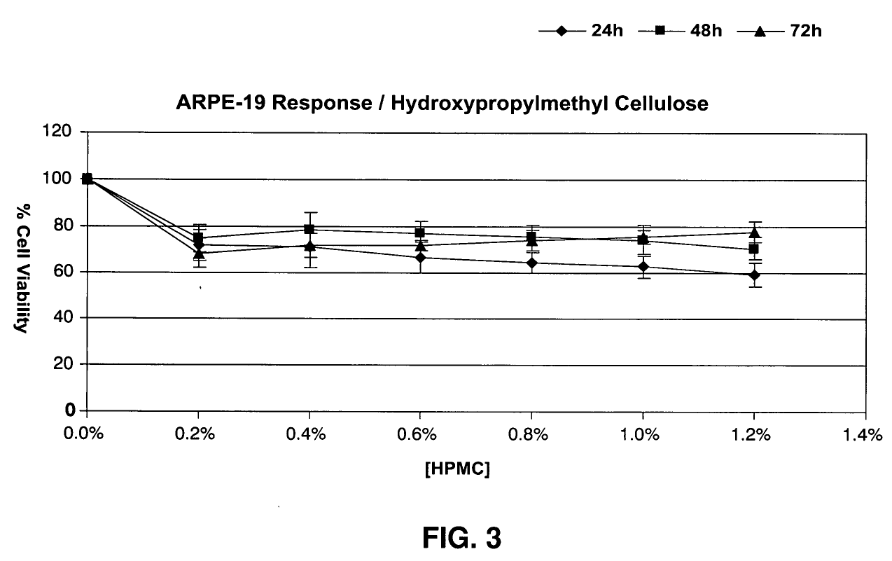

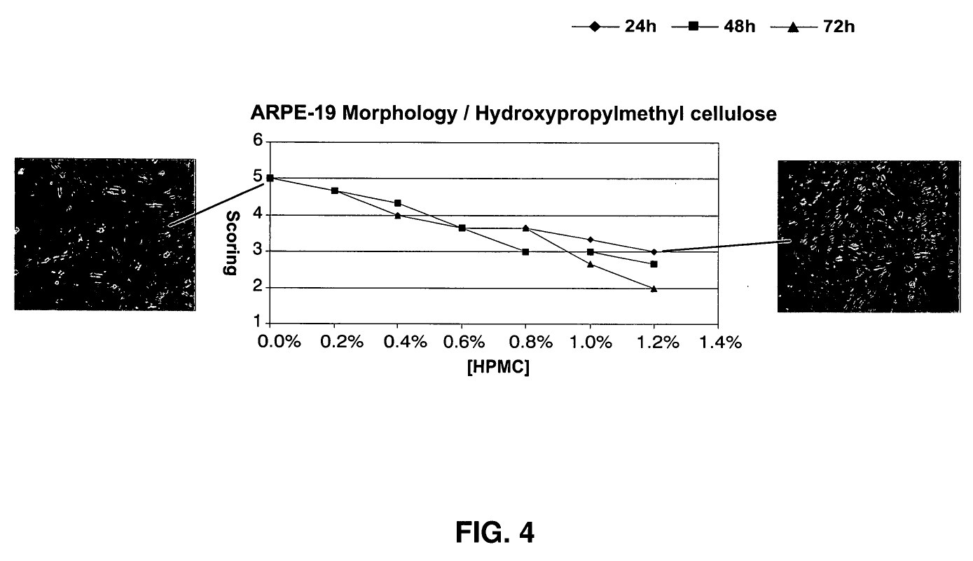

HPMC was added to ARPE-19 cultures, as described in Example 1. The cell viability results are shown in FIG. 3 and the morphology data are shown in FIG. 4. As for CMC, HPMC treatment shows a slight decrease in cell viability even at lower concentrations (FIG. 3). However, increasing concentrations of HPMC does not yield to additional noticeable dose response effects. While CMC brings percentage of cell viability comprised between 80 and 100% at all tested doses, HPMC diminishes cell viability by about 20-40% to reach 60-80% of viable cells. It is noteworthy that, similar to CMC, increasing time incubation with HPMC does not influence cell viability.

The morphological images from HPMC-treated cells show results generally comparable with those of CMC. But, different from CMC-treated cells, higher doses of HPMC (1.2%) affect cell shape less than CMC, especially for short incubation time periods (24 h) (see photographs of FIG. 4).

HPMC appears less aggressive than CMC for toxicity, based on morphological consideration, mitochondrial dehydrogenase is influenced to such an extent that overall HPMC treatment affect cells more than CMC.

The raw data for cell viability and cell morphology are provided below

Cell Viability

24 Hours

| [HPMC] (%) | 1 | 2 | 3 | Mean (%) | SD × 100 |
|---|---|---|---|---|---|
| 0 | 100.0 | 100.0 | 100.0 | 100.0 | 0 |
| 0.2 | 78.1 | 65.4 | 72.0 | 71.8 | 6.3 |
| 0.4 | 81.4 | 64.4 | 68.0 | 71.3 | 9.0 |
| 0.6 | 74.2 | 62.1 | 63.7 | 66.7 | 6.6 |
| 0.8 | 69.4 | 62.3 | 61.4 | 64.4 | 4.4 |
| 1.0 | 68.2 | 59.1 | 61.1 | 62.8 | 4.8 |
| 1.2 | 65.5 | 55.6 | 57.1 | 59.4 | 5.3 |

48 Hours

| [HPMC] (%) | 1 | 2 | 3 | Mean (%) | SD × 100 |
|---|---|---|---|---|---|
| 0 | 100.0 | 100.0 | 100.0 | 100.0 | 0 |
| 0.2 | 81.3 | 72.1 | 70.2 | 74.5 | 6.0 |
| 0.4 | 86.3 | 72.9 | 76.8 | 78.7 | 6.9 |
| 0.6 | 83.2 | 73.3 | 75.3 | 77.3 | 5.3 |
| 0.8 | 81.7 | 71.2 | 72.8 | 75.2 | 5.7 |
| 1.0 | 81.6 | 71.5 | 70.3 | 74.4 | 6.2 |
| 1.2 | 76.1 | 69.2 | 66.5 | 70.6 | 5.0 |

72 Hours

| [HPMC] (%) | 1 | 2 | 3 | Mean (%) | SD × 100 |
|---|---|---|---|---|---|
| 0 | 100.0 | 100.0 | 100.0 | 100.0 | 0 |
| 0.2 | 61.2 | 71.4 | 72.3 | 68.3 | 6.1 |
| 0.4 | 67.6 | 71.0 | 77.4 | 72.0 | 5.0 |
| 0.6 | 71.2 | 69.7 | 74.3 | 71.8 | 2.3 |
| 0.8 | 77.6 | 69.4 | 75.4 | 74.1 | 4.3 |
| 1.0 | 78.7 | 74.6 | 73.3 | 75.5 | 2.8 |
| 1.2 | 83.2 | 74.6 | 76.1 | 77.9 | 4.6 |

Cell Morphology

24 Hours

| [HPMC] (%) | 1 | 2 | 3 | Mean |
|---|---|---|---|---|
| 0 | 5 | 5 | 5 | 5.0 |
| 0.2 | 5 | 4 | 5 | 4.7 |
| 0.4 | 4 | 4 | 4 | 4.0 |
| 0.6 | 4 | 4 | 3 | 3.7 |
| 0.8 | 4 | 4 | 3 | 3.7 |
| 1.0 | 4 | 3 | 3 | 3.3 |
| 1.2 | 4 | 3 | 2 | 3.0 |

48 Hours

| [HPMC] (%) | 1 | 2 | 3 | Mean |
|---|---|---|---|---|
| 0 | 5 | 5 | 5 | 5.0 |
| 0.2 | 4 | 5 | 5 | 4.7 |
| 0.4 | 4 | 5 | 4 | 4.3 |
| 0.6 | 3 | 4 | 4 | 3.7 |
| 0.8 | 3 | 3 | 3 | 3.0 |
| 1.0 | 3 | 3 | 3 | 3.0 |
| 1.2 | 3 | 3 | 2 | 2.7 |

72 Hours

| [HPMC] (%) | 1 | 2 | 3 | Mean |
|---|---|---|---|---|
| 0 | 5 | 5 | 5 | 5.0 |
| 0.2 | 4 | 5 | 5 | 4.7 |
| 0.4 | 4 | 4 | 4 | 4.0 |
| 0.6 | 4 | 4 | 3 | 3.7 |
| 0.8 | 4 | 4 | 3 | 3.7 |
| 1.0 | 3 | 2 | 3 | 2.7 |
| 1.2 | 2 | 1 | 3 | 2.0 |

### Example 4

### Pluronic F127 Prill—Poloxamer 407 nf

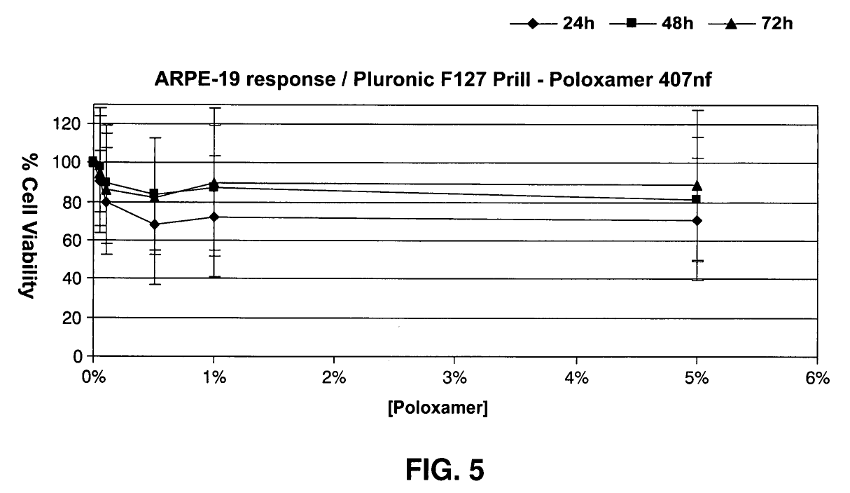

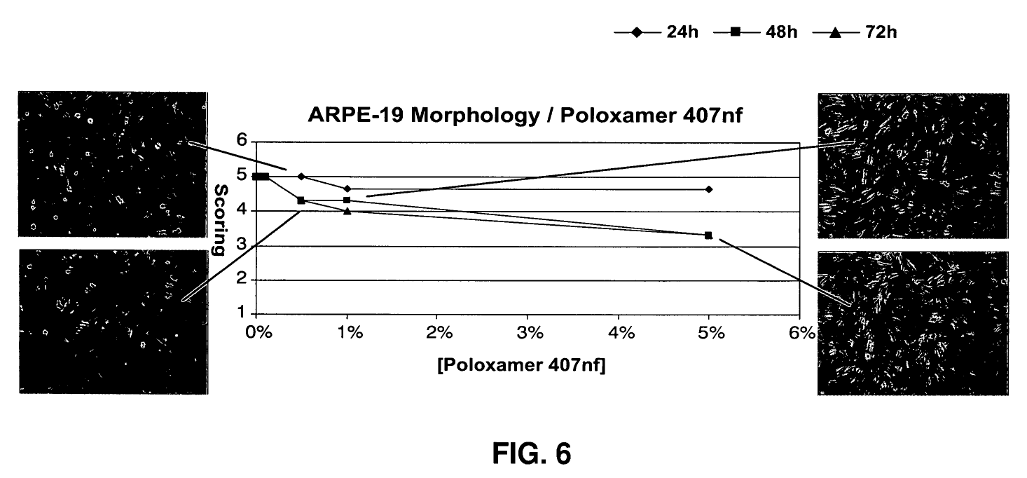

ARPE-19 cells were incubated at various time with increasing concentrations of poloxamer 407 nf from 0.1 to 10% in preliminary trial. Results are shown in FIG. 5 and FIG. 6. The results showed that 10% dose carried out surprising results probably correlated with technical issues while testing poloxamer. It appeared that even after 24 h, an increase in cell viability up to 200% was measured (data not shown).

In the light of such results, 3 hypothesis were raised. First is that cell number could double subsequently to incubation with 10% poloxamer, but considering that at beginning of experiment, cells had already reached confluence, it was rather unlikely they could have double their generation. Second, that mitochondrial metabolism is highly enhanced by treatment, bringing a higher ratio of MTT degradation and crystals formation. Third, poloxamer through non-specific binding to cells, interferes with MTT buffer at some step in the experiment, resulting in artificial coloration of cells.

During the expereiments, we noticed that variations in the rinsing step influenced the observed variations, meaning that poloxamer could possibly stick unspecifically to cell membranes or dish plastic and subsequently interact with experimental reagents (3rd hypothesis). Besides, we cannot rule out the possibility that poloxamer has an effect on mitochondrial target (2nd hypothesis), increasing enzymatic activity and directly interfering with the assay. As 10% doses gave irrepressible variations, we decided not to further perform 10% concentration in following experiments. Also, as seen in Table 2, osmolarity (462 mOsm) at this concentration far exceeded physiological value. Then, a range from 0.05 to 5% was applied in subsequent assays (FIG. 5). Nevertheless, we think that at lower doses, poloxamer also interferes with MTT assay, even to a lesser extent. This leads to high standard variation within experiment.

Cell morphology score is represented in FIG. 6. 24 h incubation with poloxamer slightly altered cell shape after 1% dose. Longer time of exposure (48 and 72 h) showed that 0.5% dose is the first concentration affecting cell shape but again, only minor changes were visible. The 5% dose resulted in a maximum modification of cell shape around 3.3 score after both 48 h and 72 h exposure.

In summary, if we consider long time exposure (72 h) to poloxamer, concentrations up to 0.1% does not show any significant changes. Doses greater than 1% modify morphological appearance of ARPE-19 cells but only to a modest extent.

The raw data for cell viability and cell morphology are provided below

Cell Viability

24 Hours

| [Poloxamer] (%) | 1 | 2 | 3 | Mean (%) | SD × 100 |
|---|---|---|---|---|---|
| 0 | 100.0 | 100.0 | 100.0 | 100.0 | 0 |
| 0.05 | 99.4 | 72.1 | 99.3 | 90.3 | 15.7 |
| 0.1 | 91.7 | 48.6 | 100.0 | 80.1 | 27.6 |
| 0.5 | 78.9 | 33.1 | 94.0 | 68.7 | 31.7 |
| 1.0 | 86.4 | 36.7 | 94.0 | 72.4 | 31.1 |
| 5.0 | 89.9 | 34.5 | 88.9 | 71.1 | 31.7 |

48 Hours

| [Poloxamer] (%) | 1 | 2 | 3 | Mean (%) | SD × 100 |
|---|---|---|---|---|---|
| 0 | 100.0 | 100.0 | 100.0 | 100.0 | 0 |
| 0.05 | 98.6 | 67.4 | 127.8 | 97.9 | 30.2 |
| 0.1 | 90.0 | 59.3 | 118.7 | 89.4 | 29.7 |
| 0.5 | 84.0 | 55.2 | 112.4 | 83.9 | 28.6 |
| 1.0 | 88.7 | 54.5 | 119.0 | 87.4 | 32.2 |
| 5.0 | 93.5 | 44.8 | 105.6 | 81.3 | 32.2 |

72 Hours

| [Poloxamer] (%) | 1 | 2 | 3 | Mean (%) | SD × 100 |
|---|---|---|---|---|---|
| 0 | 100.0 | 100.0 | 100.0 | 100.0 | 0 |
| 0.05 | 99.7 | 61.9 | 121.5 | 94.4 | 30.1 |
| 0.1 | 87.7 | 57.4 | 114.3 | 86.4 | 28.4 |
| 0.5 | 81.3 | 53.5 | 113.1 | 82.6 | 29.9 |
| 1.0 | 87.0 | 53.1 | 130.1 | 90.1 | 38.6 |
| 5.0 | 96.4 | 47.4 | 123.1 | 89.0 | 38.4 |

Cell Morphology

24 Hours

| [Poloxamer] | | | | |
|---|---|---|---|---|
| (%) | 1 | 2 | 3 | Mean |
| 0 | 5 | 5 | 5 | 5.0 |
| 0.05 | 5 | 5 | 5 | 5.0 |
| 0.1 | 5 | 5 | 5 | 5.0 |
| 0.5 | 5 | 5 | 5 | 5.0 |
| 1.0 | 5 | 5 | 4 | 4.7 |
| 5.0 | 5 | 5 | 4 | 4.7 |

48 Hours

| [Poloxamer] | | | | |
|---|---|---|---|---|
| (%) | 1 | 2 | 3 | Mean |
| 0 | 5 | 5 | 5 | 5.0 |
| 0.05 | 5 | 5 | 5 | 5.0 |
| 0.1 | 5 | 5 | 5 | 5.0 |
| 0.5 | 5 | 5 | 3 | 4.3 |
| 1.0 | 5 | 5 | 3 | 4.3 |
| 5.0 | 4 | 4 | 2 | 3.3 |

72 Hours

| [Poloxamer] | | | | |
|---|---|---|---|---|
| (%) | 1 | 2 | 3 | Mean |
| 0 | 5 | 5 | 5 | 5.0 |
| 0.05 | 5 | 5 | 5 | 5.0 |
| 0.1 | 5 | 5 | 5 | 5.0 |
| 0.5 | 5 | 5 | 3 | 4.3 |
| 1.0 | 5 | 4 | 3 | 4.0 |
| 5.0 | 5 | 4 | 1 | 3.3 |

### Example 5

### Hyaluronic Acid

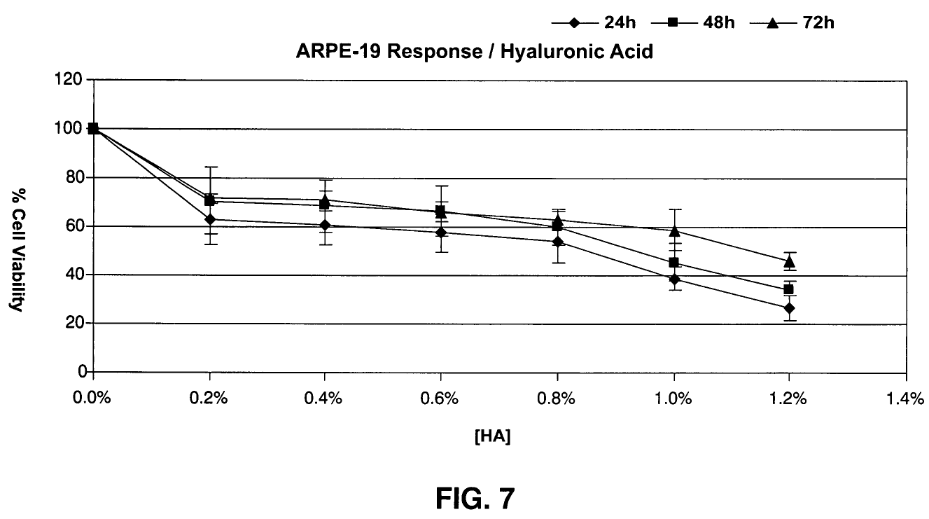

The results of the MTT assay for hyaluronic acid (FIG. 7) displayed a two phase profile. From concentrations between 0.2 to 0.6%, similar results to HPMC were obtained. From concentrations between 0.8 to 1.2%, the cell viability decreased down to nearly 25% after 24 h. Surprisingly, from concentrations between 0.8 to 1.2%, as time of exposure increased, the cells seemed to recover from treatment in a time-dependent manner. pH and osmolarity values were very similar for both excipient agents at this dose (Table 1). HA-induced cytotoxicity towards ARPE-19 cells could possibly be due to high viscosity and explain why cells react so strongly to physical constraints. In addition, or alternatively, the HA-induced cytotoxicity observed in vitro may also be related to reduced nutrient diffusion and nutrient exchange in the present of HA. For example, the HA may directly contact the ARPE-19 cells and be too sticky and thereby lead to smothering of the cultured cells.

If concentration ranges for all time points corresponding to 50% cell viability inhibition were considered, it was found that the IC₅₀ was between 0.8 and 1.1%.

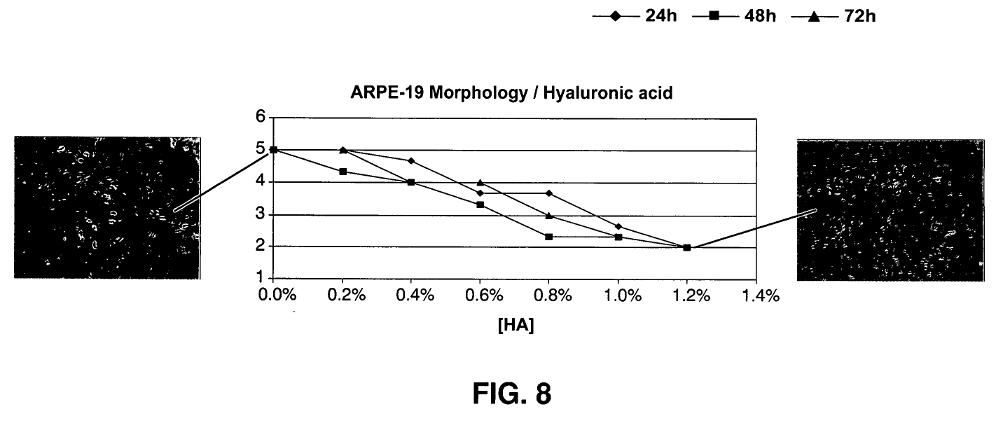

Examination of the cell morphology scoring profile in FIG. 8, showed about the same results or tendency as CMC and HPMC. The highest dose (1.2%) yielded the same score as CMC. Similar to incubation with CMC, HA-treated cells appeared as a sparse monolayer with space between cell bodies (right panel on FIG. 8).

To conclude, hyaluronic acid exerted the strongest effect on ARPE-19 cells compared to all tested viscosing agents (poloxamer 407nf, CMC, HPMC), with an IC₅₀ around 1% for all tested incubation times. Both cell viability and morphology are affected by HA treatment.

The raw data for cell viability and cell morphology are provided below

Cell Viability

24 Hours

| [HA] (%) | 1 | 2 | 3 | Mean (%) | SD × 100 |
|---|---|---|---|---|---|
| 0 | 100.0 | 100.0 | 100.0 | 100.0 | 0 |
| 0.2 | 74.1 | 60.4 | 54.5 | 63.0 | 10.0 |
| 0.4 | 70.2 | 58.1 | 53.9 | 60.7 | 8.4 |
| 0.6 | 66.7 | 55.3 | 50.9 | 57.6 | 8.1 |
| 0.8 | 63.9 | 52.9 | 46.2 | 54.3 | 8.9 |
| 1.0 | 33.4 | 39.9 | 43.1 | 38.8 | 4.9 |
| 1.2 | 32.9 | 23.3 | 24.3 | 26.8 | 5.2 |

48 Hours

| [HA] (%) | 1 | 2 | 3 | Mean (%) | SD × 100 |
|---|---|---|---|---|---|
| 0 | 100.0 | 100.0 | 100.0 | 100.0 | 0 |
| 0.2 | 70.5 | 84.2 | 56.9 | 70.5 | 13.6 |
| 0.4 | 69.6 | 78.8 | 57.6 | 68.7 | 10.7 |
| 0.6 | 65.3 | 78.0 | 57.1 | 66.8 | 10.5 |
| 0.8 | 56.7 | 68.8 | 55.0 | 60.1 | 7.6 |
| 1.0 | 36.8 | 50.3 | 49.3 | 45.4 | 7.5 |
| 1.2 | 29.9 | 37.3 | 35.2 | 34.1 | 3.8 |

72 Hours

| [HA] (%) | 1 | 2 | 3 | Mean (%) | SD × 100 |
|---|---|---|---|---|---|
| 0 | 100.0 | 100.0 | 100.0 | 100.0 | 0 |
| 0.2 | 73.6 | 71.6 | 70.0 | 71.7 | 1.8 |
| 0.4 | 75.0 | 69.9 | 67.6 | 70.8 | 3.8 |
| 0.6 | 70.5 | 65.3 | 62.4 | 66.1 | 4.1 |
| 0.8 | 61.0 | 66.9 | 61.0 | 62.9 | 3.4 |
| 1.0 | 48.7 | 63.2 | 64.1 | 58.7 | 8.6 |
| 1.2 | 41.6 | 46.7 | 49.1 | 45.8 | 3.8 |

Cell Morphology

24 Hours

| [HA] (%) | 1 | 2 | 3 | Mean |
|---|---|---|---|---|
| 0 | 5 | 5 | 5 | 5.0 |
| 0.2 | 5 | 5 | 5 | 5.0 |
| 0.4 | 5 | 4 | 5 | 4.7 |
| 0.6 | 3 | 4 | 4 | 3.7 |
| 0.8 | 3 | 4 | 4 | 3.7 |
| 1.0 | 2 | 3 | 3 | 2.7 |
| 1.2 | 2 | 2 | 2 | 2.0 |

48 Hours

| [HA] (%) | 1 | 2 | 3 | Mean |
|---|---|---|---|---|
| 0 | 5 | 5 | 5 | 5.0 |
| 0.2 | 4 | 4 | 5 | 4.3 |
| 0.4 | 4 | 3 | 5 | 4.0 |
| 0.6 | 3 | 3 | 4 | 3.3 |
| 0.8 | 2 | 2 | 3 | 2.3 |
| 1.0 | 2 | 2 | 2 | 2.3 |
| 1.2 | 2 | 2 | 2 | 2.0 |

72 Hours

| [HA] (%) | 1 | 2 | 3 | Mean |
|---|---|---|---|---|
| 0 | 5 | 5 | 5 | 5.0 |
| 0.2 | 5 | 5 | 5 | 5.0 |
| 0.4 | 3 | 4 | 5 | 4.0 |
| 0.6 | 3 | 4 | 5 | 4.0 |
| 0.8 | 2 | 3 | 4 | 3.0 |
| 1.0 | 2 | 2 | 3 | 2.3 |
| 1.2 | 2 | 2 | 2 | 2.0 |

### Example 6

### Hydroxypropyl Gamma-CD (Cavasol®)

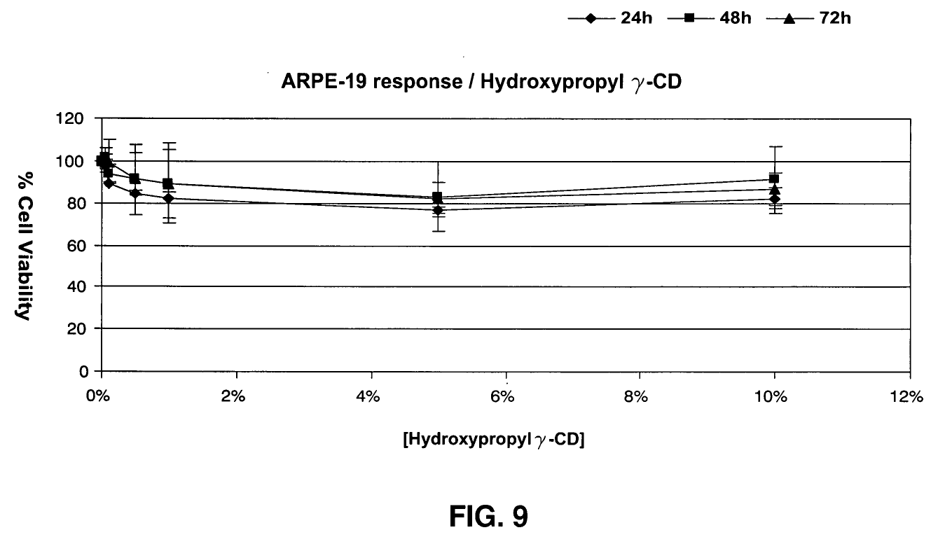

ARPE-19 cells were treated with increasing concentrations of Cavasol® (from 0.05 to 10%) during 24, 48 and 72 h. Cell viability results are presented in FIG. 9. It was observed that Cavasol® slightly decreased ARPE-19 viability for any tested concentration. A small decrease was noticeable after 24 h (˜80% viable cells), and then, cells seemed to recover to maintain a percentage of living cells greater than approximately 80%. Therefore, incubation time does not seem to influence the cytotoxic effect of Cavasol® on ARPE-19 cells.

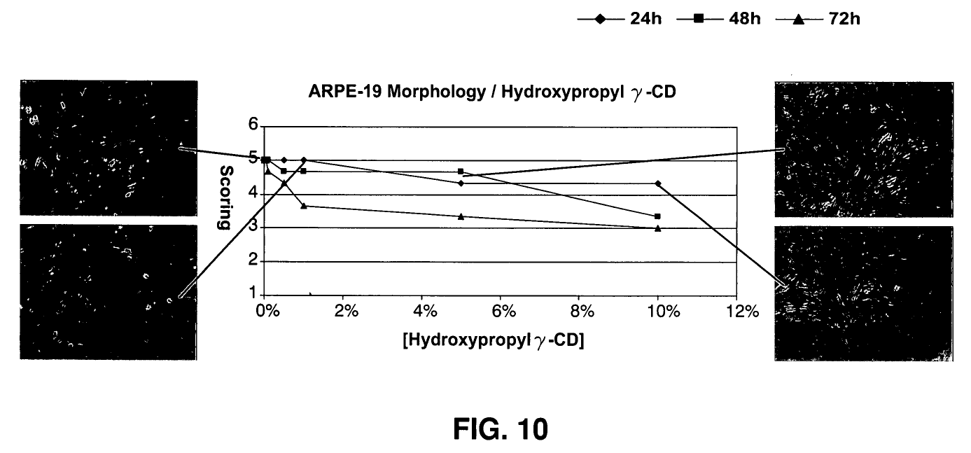

ARPE-19 cell shape was visualized using light microscopy and scored semi-quantitatively. Results are presented in FIG. 10.

We observed that a 24 hour incubation did not seem to affect cell morphology, even at a 10% concentration. At the 48 hour time point, treatment at 10% cyclodextrin did not greatly alter cell shape. On the contrary, 72 h of incubation showed slight to moderate morphological changes from 0.1 to 10% respectively. Therefore, 72 h incubation appeared to impact cell shape more than either 24 or 48 h time of exposure.

Besides these morphological changes, the MTT assay did not reflect a strong modification in mitochondrial metabolism resulting from contacting the ARPE-19 cells with Cavasol®. It was concluded that Cavasol® has an overall limited cytotoxicity to ARPE-19 cells since it was not possible to determine the IC₅₀ at the tested concentrations and incubation times.

The raw data for cell viability and cell morphology are provided below

Cell Viability

24 Hours

| [Cavasol] | | | | | |
|---|---|---|---|---|---|
| (%) | 1 | 2 | 3 | Mean (%) | SD × 100 |
| 0 | 100.0 | 100.0 | 100.0 | 100.0 | 0 |
| 0.05 | 93.9 | 100.6 | 98.2 | 97.6 | 3.4 |
| 0.1 | 89.3 | 89.0 | 89.8 | 89.3 | 0.4 |
| 0.5 | 83.1 | 85.2 | 85.5 | 84.6 | 1.3 |
| 1.0 | 79.2 | 84.1 | 83.2 | 82.2 | 2.6 |
| 5.0 | 75.3 | 78.2 | 77.0 | 76.9 | 1.5 |
| 10.0 | 81.4 | 87.6 | 78.2 | 82.4 | 4.8 |

48 Hours

| [Cavasol] | | | | | |
|---|---|---|---|---|---|
| (%) | 1 | 2 | 3 | Mean (%) | SD × 100 |
| 0 | 100.0 | 100.0 | 100.0 | 100.0 | 0 |
| 0.05 | 100.5 | 106.0 | 97.0 | 101.1 | 4.6 |
| 0.1 | 95.4 | 88.3 | 97.3 | 93.6 | 4.8 |
| 0.5 | 87.0 | 82.3 | 105.2 | 91.5 | 12.1 |
| 1.0 | 86.0 | 74.9 | 106.5 | 89.1 | 16.1 |
| 5.0 | 94.0 | 64.1 | 91.2 | 83.1 | 16.5 |
| 10.0 | 109.0 | 79.7 | 84.7 | 91.1 | 15.7 |

72 Hours

| [Cavasol] | | | | | |
|---|---|---|---|---|---|
| (%) | 1 | 2 | 3 | Mean (%) | SD × 100 |
| 0 | 100.0 | 100.0 | 100.0 | 100.0 | 0 |
| 0.05 | 98.3 | 103.2 | 96.5 | 99.3 | 3.4 |
| 0.1 | 89.9 | 97.3 | 110.4 | 99.2 | 10.4 |
| 0.5 | 79.1 | 84.5 | 110.0 | 91.2 | 16.5 |
| 1.0 | 75.0 | 81.8 | 110.8 | 89.2 | 19.0 |
| 5.0 | 72.9 | 84.6 | 88.3 | 81.9 | 8.0 |
| 10.0 | 94.8 | 86.0 | 79.4 | 86.7 | 7.8 |

Cell Morphology

24 Hours

| [Cavasol] | | | | |
|---|---|---|---|---|
| (%) | 1 | 2 | 3 | Mean |
| 0 | 5 | 5 | 5 | 5.0 |
| 0.05 | 5 | 5 | 5 | 5.0 |
| 0.1 | 5 | 5 | 5 | 5.0 |
| 0.5 | 5 | 5 | 5 | 5.0 |
| 1.0 | 5 | 5 | 5 | 5.0 |
| 5.0 | 5 | 5 | 3 | 4.3 |
| 10.0 | 5 | 5 | 3 | 4.3 |

48 Hours

| [Cavasol] | | | | |
|---|---|---|---|---|
| (%) | 1 | 2 | 3 | Mean |
| 0 | 5 | 5 | 5 | 5.0 |
| 0.05 | 5 | 5 | 5 | 5.0 |
| 0.1 | 5 | 5 | 5 | 5.0 |
| 0.5 | 5 | 5 | 4 | 4.7 |
| 1.0 | 5 | 5 | 4 | 4.7 |
| 5.0 | 5 | 5 | 4 | 4.7 |
| 10.0 | 5 | 4 | 1 | 3.3 |

72 Hours

| [Cavasol] | | | | |
|---|---|---|---|---|
| (%) | 1 | 2 | 3 | Mean |
| 0 | 5 | 5 | 5 | 5.0 |
| 0.05 | 5 | 5 | 5 | 5.0 |
| 0.1 | 5 | 5 | 4 | 4.7 |
| 0.5 | 5 | 5 | 3 | 4.3 |
| 1.0 | 5 | 5 | 1 | 3.7 |
| 5.0 | 5 | 4 | 1 | 3.3 |
| 10.0 | 4 | 4 | 1 | 3.0 |

### Example 7

### Sulfobutyl Ether 4 Beta-CD (Captisol®)

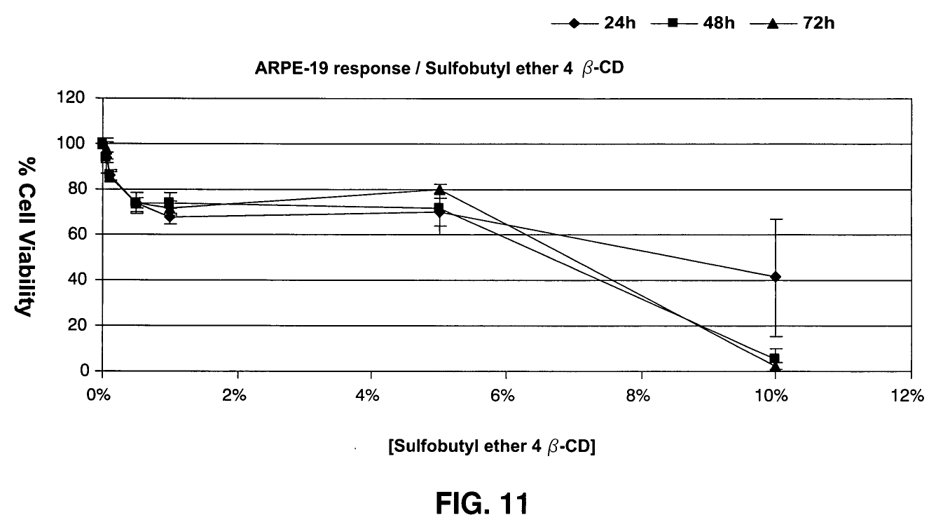

As with Cavsol®, ARPE-19 cells were incubated at various times with increasing concentrations of Captisol® (from 0.05 to 10%). FIG. 11 represents MTT assay results. It was first deduced that increasing incubation time does not appear to enhance Captisol®-induced cytotoxicity, except for 10% concentration. 24 h of incubation at this dose resulted in a decrease to only 40% cell viability compared to 48 and 72 h treatment which lead to complete lethality. From the profiles for all the incubation times, the IC₅₀ was deduced and determined to be between 6.5 and 8.5%.

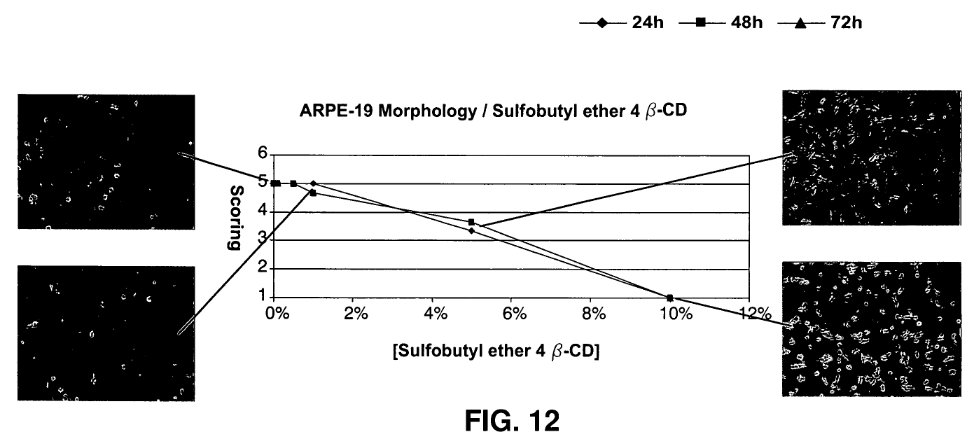

Morphological appearance scoring showed comparable curves at all incubation times (FIG. 12). From a 1% dose, an alteration (although slight at 24 h) of cell shape was apparent. This alteration correlated with the cell viability assay data. At the 5% dose, the cell shape was moderately altered. The 5% concentration corresponded to the inferior limit concentration bringing to 50% cell death determined through the MTT assay (FIG. 11). On this graph, the apparent critical limit concentration was around 6%, which may be related to exceeding the tonicity of tested composition.

It was concluded that sulfobutyl ether 4 beta-CD-Captisol® showed a mild cytotoxicity to retinal cells. Although a lethal cytotoxic effect on cells was detected, the effect was noticeable at concentrations greater than 5% which far exceeds usually used cyclodextrin concentration in formulations. Sulfobutyl ether 4 beta-CD appeared to affect cell metabolism and shape in a time-independent manner.

The raw data for cell viability and cell morphology are provided below

Cell Viability

24 Hours

| [Captisol] | | | | | |
|---|---|---|---|---|---|
| (%) | 1 | 2 | 3 | Mean (%) | SD × 100 |
| 0 | 100.0 | 100.0 | 100.0 | 100.0 | 0 |
| 0.05 | 96.4 | 94.3 | 91.6 | 94.1 | 2.4 |
| 0.1 | 89.0 | 83.7 | 85.2 | 86.0 | 2.7 |
| 0.5 | 78.9 | 70.6 | 71.2 | 73.6 | 4.7 |
| 1.0 | 72.3 | 65.7 | 65.9 | 68.0 | 3.7 |
| 5.0 | 77.3 | 67.1 | 65.7 | 70.0 | 6.3 |
| 10.0 | 62.9 | 48.1 | 12.5 | 41.2 | 25.9 |

48 Hours

| [Captisol] | | | | | |
|---|---|---|---|---|---|
| (%) | 1 | 2 | 3 | Mean (%) | SD × 100 |
| 0 | 100.0 | 100.0 | 100.0 | 100.0 | 0 |
| 0.05 | 97.2 | 98.4 | 86.2 | 93.9 | 6.8 |
| 0.1 | 88.2 | 85.5 | 84.4 | 86.0 | 2.0 |
| 0.5 | 78.7 | 73.2 | 70.7 | 74.2 | 4.1 |
| 1.0 | 73.2 | 69.5 | 78.8 | 73.8 | 4.7 |
| 5.0 | 78.6 | 77.1 | 58.5 | 71.4 | 11.2 |
| 10.0 | 10.3 | 5.2 | 0.9 | 5.5 | 4.7 |

72 Hours

| [Captisol] | | | | | |
|---|---|---|---|---|---|
| (%) | 1 | 2 | 3 | Mean (%) | SD × 100 |
| 0 | 100.0 | 100.0 | 100.0 | 100.0 | 0 |
| 0.05 | 102.7 | 96.1 | 94.0 | 97.6 | 4.5 |
| 0.1 | 87.5 | 82.5 | 87.0 | 85.7 | 2.7 |
| 0.5 | 76.6 | 72.2 | 72.5 | 73.8 | 2.5 |
| 1.0 | 75.1 | 69.8 | 70.1 | 71.7 | 3.0 |
| 5.0 | 81.8 | 78.0 | 80.7 | 80.2 | 2.0 |
| 10.0 | 3.7 | 1.4 | 0.9 | 2.0 | 1.5 |

Cell Morphology

24 Hours

| [Captisol] | | | | |
|---|---|---|---|---|
| (%) | 1 | 2 | 3 | Mean |
| 0 | 5 | 5 | 5 | 5.0 |
| 0.05 | 5 | 5 | 5 | 5.0 |
| 0.1 | 5 | 5 | 5 | 5.0 |
| 0.5 | 5 | 5 | 5 | 5.0 |
| 1.0 | 5 | 5 | 5 | 5.0 |
| 5.0 | 4 | 2 | 4 | 3.3 |
| 10.0 | 1 | 1 | 1 | 1.0 |

48 Hours

| [Captisol] | | | | |
|---|---|---|---|---|
| (%) | 1 | 2 | 3 | Mean |
| 0 | 5 | 5 | 5 | 5.0 |
| 0.05 | 5 | 5 | 5 | 5.0 |
| 0.1 | 5 | 5 | 5 | 5.0 |
| 0.5 | 5 | 5 | 5 | 5.0 |
| 1.0 | 5 | 5 | 4 | 4.7 |
| 5.0 | 4 | 4 | 3 | 3.7 |
| 10.0 | 1 | 1 | 1 | 1.0 |

72 Hours

| [Captisol] | | | | |
|---|---|---|---|---|
| (%) | 1 | 2 | 3 | Mean |
| 0 | 5 | 5 | 5 | 5.0 |
| 0.05 | 5 | 5 | 5 | 5.0 |
| 0.1 | 5 | 5 | 5 | 5.0 |
| 0.5 | 5 | 5 | 5 | 5.0 |
| 1.0 | 4 | 5 | 5 | 4.7 |
| 5.0 | 4 | 4 | 3 | 3.7 |
| 10.0 | 1 | 1 | 1 | 1.0 |

### Example 8

### Hydroxypropyl Beta-CD (Kleptose®)

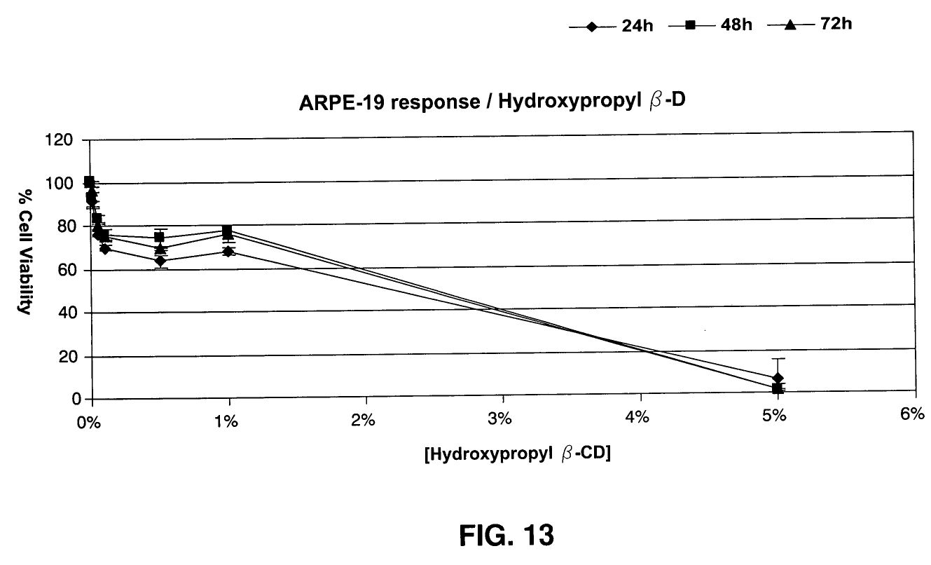

Kleptose® was evaluated in the same manner as the previous agents but at lower minimum and maximum doses (0.01% and 5%, respectively) upon results from preliminary studies (10% dose was as lethal as 5% dose). FIG. 13 shows the MTT assay results. For concentrations below 1%, cell viability decreased by about 40% (like sulfobutyl ether 4 beta-CD). On the contrary to the cyclodextrins of Examples 6 and 7, the 5% dose of Kleptose® exhibited a harsh cytotoxic effect on ARPE-19 since 24 h of incubation lead to complete disappearance of living cells. The IC₅₀ is determined to be about 2.2%.

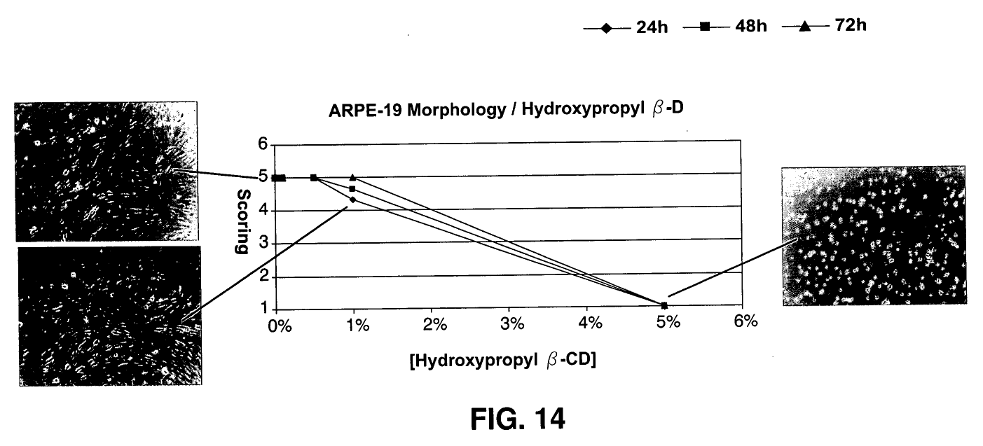

Cell morphology was followed and scored. As shown in FIG. 14, cell shape was not altered for concentrations of Kleptose® below 1%. For greater doses, morphology was slightly altered at 1% and dramatically evolved to lethal phenotype at 5%. Even if mitochondrial metabolism seemed to be sensitive to concentration as low as 0.1% of Kleptose® (see FIG. 13), cell shape did not show variations at such concentrations. At 0.5%, cell shape remained visibly normal while cell viability decreased to about 63% viable cells after 24 h incubation. Morphological appearance correlated with cell viability for concentrations over 1%.

From our data, it was concluded that hydroxypropyl beta-CD has an IC₅₀ around 2.5% independently from time of exposure.

The raw data for cell viability and cell morphology are provided below

Cell Viability

24 Hours

| [Kleptose] | | | | | |
|---|---|---|---|---|---|
| (%) | 1 | 2 | 3 | Mean (%) | SD × 100 |
| 0 | 100.0 | 100.0 | 100.0 | 100.0 | 0 |
| 0.01 | 90.1 | 95.8 | 89.0 | 91.6 | 3.7 |
| 0.05 | 74.7 | 77.5 | 76.7 | 76.3 | 1.4 |
| 0.1 | 67.9 | 70.6 | 70.5 | 69.7 | 1.5 |
| 0.5 | 60.5 | 66.4 | 63.5 | 63.5 | 3.0 |
| 1.0 | 66.8 | 66.4 | 69.9 | 67.7 | 1.9 |
| 5.0 | 16.9 | 1.3 | 0.2 | 6.1 | 9.4 |

48 Hours

| [Kleptose] (%) | 1 | 2 | 3 | Mean (%) | SD × 100 |
|---|---|---|---|---|---|
| 0 | 100.0 | 100.0 | 100.0 | 100.0 | 0 |
| 0.01 | 94.8 | 96.6 | 88.3 | 93.2 | 4.4 |
| 0.05 | 83.6 | 84.7 | 81.0 | 83.1 | 1.9 |
| 0.1 | 78.6 | 75.6 | 72.9 | 75.7 | 2.9 |
| 0.5 | 79.0 | 70.7 | 72.6 | 74.1 | 4.4 |
| 1.0 | 76.9 | 75.5 | 80.4 | 77.6 | 2.5 |
| 5.0 | 4.1 | 0.9 | 0.5 | 1.9 | 2.0 |

72 Hours

| [Kleptose] | | | | | |
|---|---|---|---|---|---|
| (%) | 1 | 2 | 3 | Mean (%) | SD × 100 |
| 0 | 100.0 | 100.0 | 100.0 | 100.0 | 0 |
| 0.01 | 94.2 | 101.1 | 92.8 | 96.1 | 4.5 |
| 0.05 | 78.2 | 81.9 | 80.1 | 80.1 | 1.8 |
| 0.1 | 74.5 | 76.1 | 74.2 | 74.9 | 1.1 |
| 0.5 | 70.1 | 68.8 | 69.3 | 69.4 | 0.7 |
| 1.0 | 73.4 | 73.0 | 80.2 | 75.6 | 4.1 |
| 5.0 | 1.5 | 1.9 | 1.1 | 1.5 | 0.4 |

Cell Morphology

24 Hours

| [Kleptose] | | | | |
|---|---|---|---|---|
| (%) | 1 | 2 | 3 | Mean |
| 0 | 5 | 5 | 5 | 5.0 |
| 0.01 | 5 | 5 | 5 | 5.0 |
| 0.05 | 5 | 5 | 5 | 5.0 |
| 0.1 | 5 | 5 | 5 | 5.0 |
| 0.5 | 5 | 5 | 5 | 5.0 |
| 1.0 | 5 | 4 | 4 | 4.3 |
| 5.0 | 1 | 1 | 1 | 1.0 |

48 Hours

| [Kleptose] | | | | |
|---|---|---|---|---|
| (%) | 1 | 2 | 3 | Mean |
| 0 | 5 | 5 | 5 | 5.0 |
| 0.01 | 5 | 5 | 5 | 5.0 |
| 0.05 | 5 | 5 | 5 | 5.0 |
| 0.1 | 5 | 5 | 5 | 5.0 |
| 0.5 | 5 | 5 | 5 | 5.0 |
| 1.0 | 5 | 5 | 4 | 4.7 |
| 5.0 | 1 | 1 | 1 | 1.0 |

72 Hours

| [Kleptose] | | | | |
|---|---|---|---|---|
| (%) | 1 | 2 | 3 | Mean |
| 0 | 5 | 5 | 5 | 5.0 |
| 0.01 | 5 | 5 | 5 | 5.0 |
| 0.05 | 5 | 5 | 5 | 5.0 |
| 0.1 | 5 | 5 | 5 | 5.0 |
| 0.5 | 5 | 5 | 5 | 5.0 |
| 1.0 | 5 | 5 | 5 | 5.0 |
| 5.0 | 1 | 1 | 1 | 1.0 |

### Example 9

### Benzyl Alcohol (BOH)

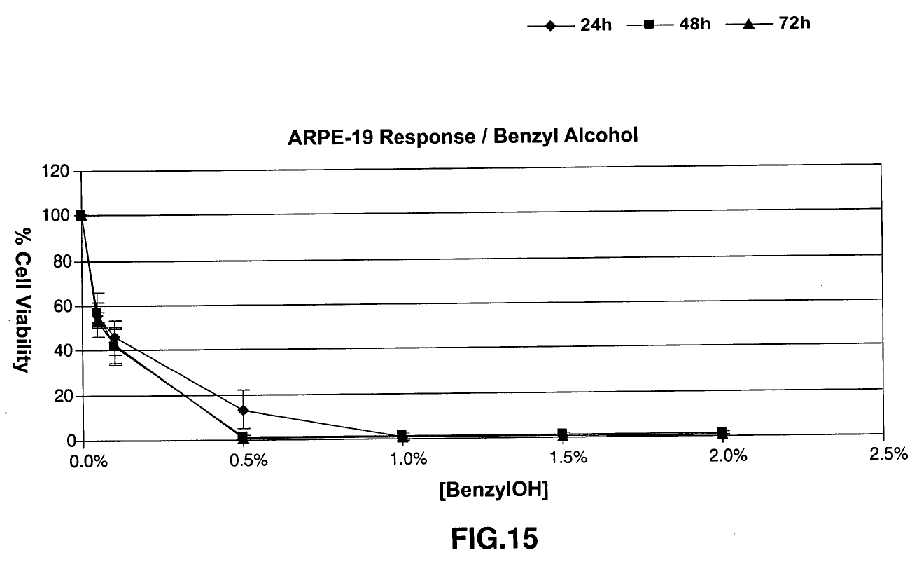

Benzyl alcohol-induced cytotoxicity on ARPE-19 cells was assessed using the methods described above. Concentrations from 0.05 to 2% were applied to ARPE-19 cell cultures. FIG. 15 represents MTT assay results obtained for 24 to 72 h time of exposure. The lowest concentration tested (i.e., 0.05%) at all incubation times resulted in a decrease of about 45% in cell viability. This dramatic effect was evident at concentrations up to 0.5% where living cells were not visible. This profile is almost independent of time of exposure, as 24 h gives maximal effect (except for 0.5% at 24 h). Based on the cell viability profile shown in FIG. 15, the IC₅₀ for benzyl alcohol was about 0.07%.

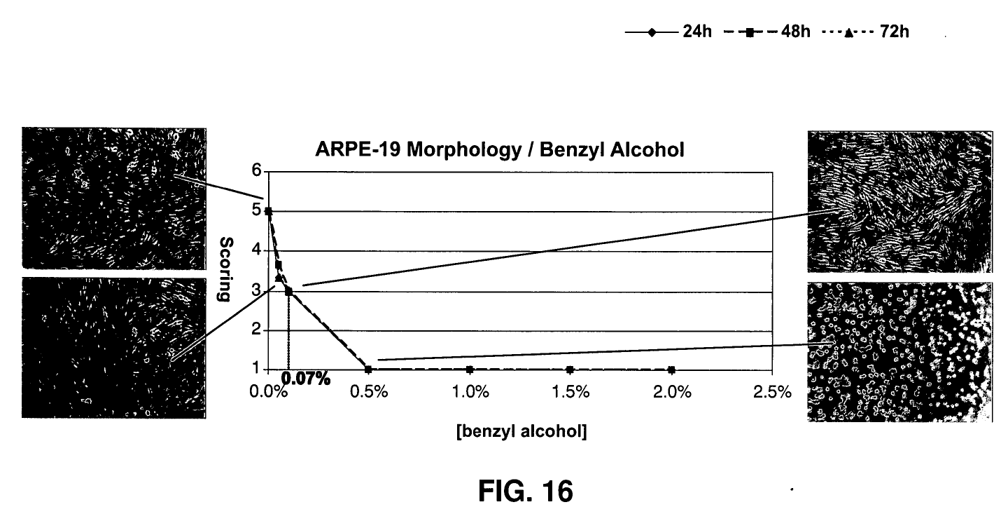

The IC₅₀ for benzyl alcohol also appears to be about 0.07% when cell morphology is examined, as shown in FIG. 16. At a 0.1% dose, cells appeared stressed and exhibited long typical phenotypes (see panel on FIG. 16) At 0.5% concentrations of benzyl alcohol, no living cells survived.

As a whole these results demonstrate that ARPE-19 cells are particularly sensitive to benzyl alcohol. Even at concentrations as low as 0.05%, an intense impact is measured both on cell viability and morphological aspect. The IC₅₀ deduced from the graphs is approximately around 0.07% for every tested time of incubation.

The raw data for cell viability and cell morphology are provided below

Cell Viability

24 Hours

| [BOH] (%) | 1 | 2 | 3 | Mean (%) | SD × 100 |
|---|---|---|---|---|---|
| 0 | 100.0 | 100.0 | 100.0 | 100.0 | 0 |
| 0.05 | 56.1 | 45.6 | 65.4 | 55.7 | 9.9 |
| 0.1 | 48.0 | 37.5 | 52.2 | 45.9 | 7.6 |
| 0.5 | 23.0 | 9.7 | 7.3 | 13.3 | 8.5 |
| 1.0 | 0.1 | 0.8 | 0.2 | 0.4 | 0.3 |
| 1.5 | 0.2 | 0.7 | 0.2 | 0.4 | 0.3 |
| 2.0 | 0.1 | 0.7 | 0.1 | 0.3 | 0.3 |

48 Hours

| [BOH] (%) | 1 | 2 | 3 | Mean (%) | SD × 100 |
|---|---|---|---|---|---|
| 0 | 100.0 | 100.0 | 100.0 | 100.0 | 0 |
| 0.05 | 60.9 | 52.4 | 58.6 | 57.3 | 4.4 |
| 0.1 | 49.0 | 33.8 | 43.2 | 42.0 | 7.7 |
| 0.5 | 1.2 | 2.0 | 1.1 | 1.5 | 0.5 |
| 1.0 | 0.7 | 2.9 | 0.8 | 1.5 | 1.2 |
| 1.5 | 0.4 | 2.7 | 0.7 | 1.3 | 1.2 |
| 2.0 | 0.3 | 2.4 | 0.8 | 1.2 | 1.1 |

72 Hours

| [BOH] (%) | 1 | 2 | 3 | Mean (%) | SD × 100 |
|---|---|---|---|---|---|
| 0 | 100.0 | 100.0 | 100.0 | 100.0 | 0 |
| 0.05 | 57.5 | 52.9 | 50.8 | 53.7 | 3.4 |
| 0.1 | 49.1 | 44.9 | 32.6 | 42.2 | 8.6 |
| 0.5 | 2.6 | 0.2 | 0.3 | 1.1 | 1.4 |
| 1.0 | 2.4 | 0.2 | 0.1 | 0.9 | 1.3 |
| 1.5 | 2.2 | 0.4 | 0.3 | 1.0 | 1.0 |
| 2.0 | 2.0 | 0.5 | 0.4 | 1.0 | 0.9 |

Cell Morphology

24 Hours

| [BOH] (%) | 1 | 2 | 3 | Mean |
|---|---|---|---|---|
| 0 | 5 | 5 | 5 | 5.0 |
| 0.05 | 3 | 3 | 4 | 3.3 |
| 0.1 | 3 | 3 | 3 | 3.0 |
| 0.5 | 1 | 1 | 1 | 1.0 |
| 1.0 | 1 | 1 | 1 | 1.0 |
| 1.5 | 1 | 1 | 1 | 1.0 |
| 2.0 | 1 | 1 | 1 | 1.0 |

48 Hours

| [BOH] (%) | 1 | 2 | 3 | Mean |
|---|---|---|---|---|
| 0 | 5 | 5 | 5 | 5.0 |
| 0.05 | 3 | 4 | 4 | 3.7 |
| 0.1 | 3 | 3 | 3 | 3.0 |
| 0.5 | 1 | 1 | 1 | 1.0 |
| 1.0 | 1 | 1 | 1 | 1.0 |
| 1.5 | 1 | 1 | 1 | 1.0 |
| 2.0 | 1 | 1 | 1 | 1.0 |

72 Hours

| [BOH] (%) | 1 | 2 | 3 | Mean |
|---|---|---|---|---|
| 0 | 5 | 5 | 5 | 5.0 |
| 0.05 | 3 | 3 | 4 | 3.3 |
| 0.1 | 3 | 3 | 3 | 3.0 |
| 0.5 | 1 | 1 | 1 | 1.0 |
| 1.0 | 1 | 1 | 1 | 1.0 |
| 1.5 | 1 | 1 | 1 | 1.0 |
| 2.0 | 1 | 1 | 1 | 1.0 |

### Example 10

### Borate Buffer

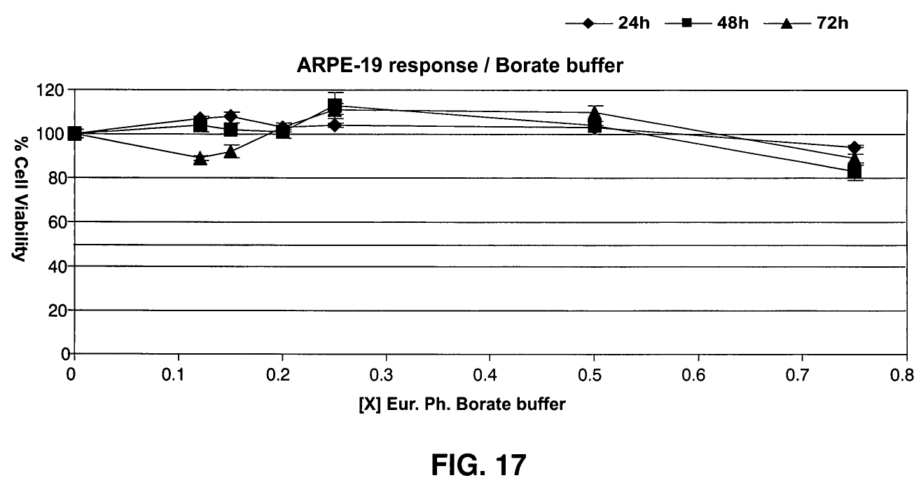

The effects of borate buffer were also tested on ARPE-19 cell viability and cell morphology. ARPE-19 cells were treated with a range of concentration from 0.12 to 0.75 times the concentration of borate buffer prepared according to European Pharmacopeia. FIG. 17 illustrates the results obtained from one experiment of an MTT assay for borate buffer. The data demonstrate that borate buffer barely affects mitochondrial metabolism. Low concentrations (0.12-0.25) faintly modified cell metabolism. Higher concentrations (up to 0.75) resulted in a decrease of cell viability less than 20%.

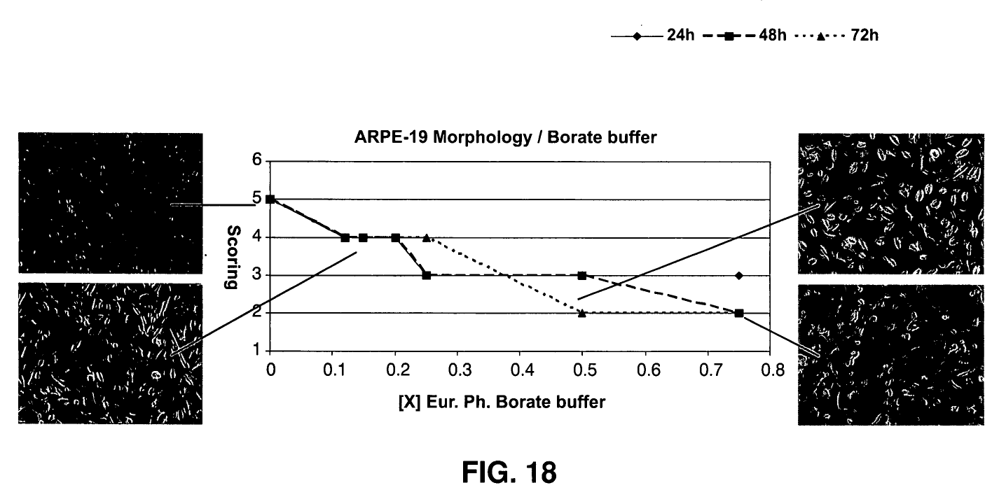

Morphological scoring for borate buffer is shown in FIG. 18. FIG. 18 shows that very low concentrations of borate buffer altered cell shape to a small extent 0.12 to 0.2 (below left panel on FIG. 18). From 0.25 to 0.5 concentrations, the cell aspect undergoes more changes, and from 0.5 to 0.75 large areas of the culture were devoid of attached cells after 72 h incubation (see upper right panel on FIG. 18). Although cell metabolism seemed poorly affected by treatment with borate buffer, cell shape exhibited substantial changes as both density and morphology of cells vary upon incubation.

Based on these results, it was concluded that borate buffer seems well accepted by ARPE-19 cells under the experimental conditions in vitro. A time dependent effect was seen in morphology even if cell viability did not seem to be greatly affected at the tested concentrations. In these experimental conditions, it was not possible to presume any IC₅₀ values.

The raw data for cell viability and cell morphology are provided below

Cell Vitality

24 Hours

| [Eur. Phar. | |
|---|---|
| Borate | |
| Buffer] (%) | (%) |
| 0 | 100.0 |
| 0.12 | 106.9 |
| 0.15 | 107.7 |
| 0.2 | 102.9 |
| 0.25 | 104.0 |
| 0.5 | 103.4 |
| 0.75 | 94.1 |

48 Hours

| [Eur. Phar. | |
|---|---|
| Borate | |
| Buffer] (%) | (%) |
| 0 | 100.0 |
| 0.12 | 104.5 |
| 0.15 | 102.4 |
| 0.2 | 101.0 |
| 0.25 | 113.1 |
| 0.5 | 104.3 |
| 0.75 | 82.9 |

72 Hours

| [Eur. Phar. | |
|---|---|
| Borate | |
| Buffer] (%) | (%) |
| 0 | 100.0 |
| 0.12 | 89.0 |
| 0.15 | 92.1 |
| 0.2 | 103.1 |
| 0.25 | 111.2 |
| 0.5 | 109.8 |
| 0.75 | 88.8 |

Cell Morphology

24 Hours

| [Eur. Phar. | |
|---|---|
| Borate | |
| Buffer] (%) | Score |
| 0 | 5 |
| 0.12 | 4 |
| 0.15 | 4 |
| 0.2 | 4 |
| 0.25 | 3 |
| 0.5 | 3 |
| 0.75 | 3 |

48 Hours

| [Eur. Phar. | |
|---|---|
| Borate | |
| Buffer] (%) | Score |
| 0 | 5 |
| 0.12 | 4 |
| 0.15 | 4 |
| 0.2 | 4 |
| 0.25 | 3 |
| 0.5 | 3 |
| 0.75 | 2 |

72 Hours

| [Eur. Phar. | |
|---|---|
| Borate | |
| Buffer] (%) | Score |
| 0 | 5 |
| 0.12 | 4 |
| 0.15 | 4 |
| 0.2 | 4 |
| 0.25 | 4 |
| 0.5 | 2 |
| 0.75 | 2 |

### Example 11

### Phosphate Buffer (X)

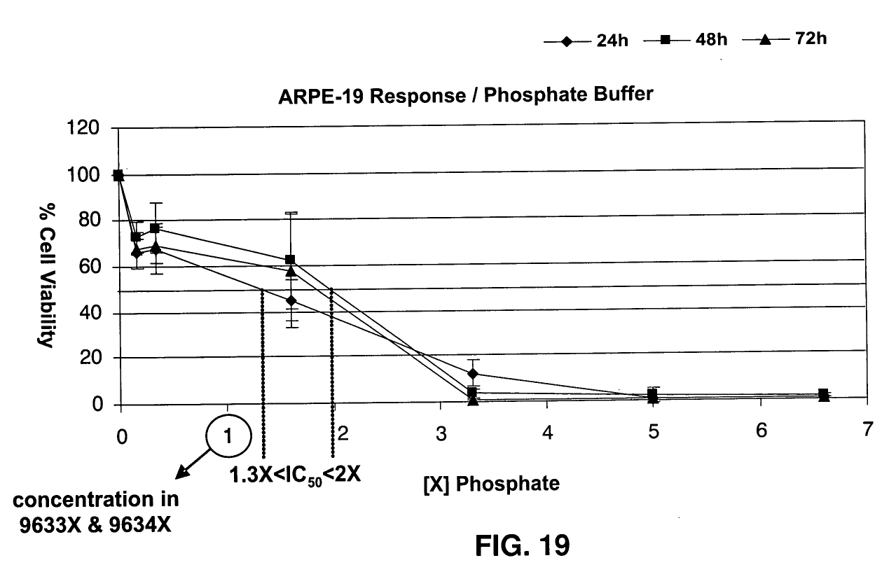

Phosphate buffer was tested on human retinal cells as described above. FIG. 19 shows an almost complete inhibition of formazan crystal formation for 3.3× dose (e.g., a reduction in cell viability about 100%). Cell viability moderately decreased for low concentrations, such as 0.16× and 0.33×. For a 1.6× dose, a significant decrease in cell viability was observed, around 40˜60% of viable cells (e.g., a 40%-60% reduction in cell viability). The IC₅₀ was deduced from the data in FIG. 19 and was determined to be between 1.3× and 2×. One explanation for these results may be related to a calcium imbalance. For example, in vitro calcium may form a precipatation resulting in no free calcium at high phosphate concentrations.

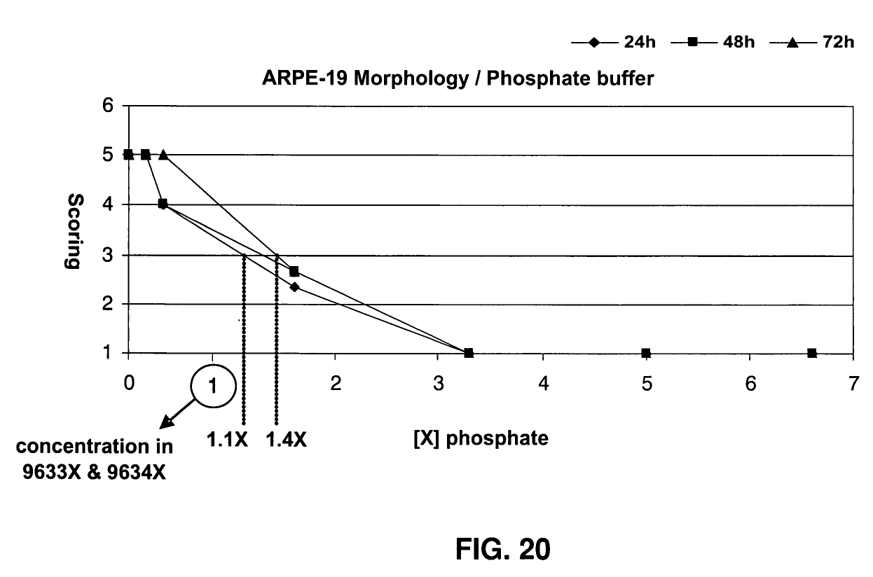

Morphological scores for phosphate buffer treatment are shown in FIG. 20. In FIG. 20, we observed that doses of 1.6× altered morphology in a non negligible manner, and even after 24 h time of exposure. Additional incubations did not affect the outcome. Treatment for 24 h with 3.3× phosphate buffer resulted in extreme phenotype where living cells were not observed. Critical limit concentration for morphology integrity was estimated to be about 1.1×.

These results show that the metabolic effects and morphological effects caused by phosphate buffer are similar. The critical limits appeared to be higher than concentrations of phosphate buffer found in existing ophthalmic formulations, especially concerning cell viability parameter.

The raw data for cell viability and cell morphology are provided below

Cell Viability

24 Hours

| [X] | 1 | 2 | 3 | Mean (%) | SD × 100 |
|---|---|---|---|---|---|
| 0 | 100.0 | 100.0 | 100.0 | 100.0 | 0 |
| 0.16 | 59.1 | 66.9 | 71.5 | 65.9 | 6.3 |
| 0.33 | 57.9 | 66.2 | 79.1 | 67.7 | 10.7 |
| 1.6 | 52.9 | 47.2 | 35.0 | 45.0 | 9.1 |
| 3.3 | 8.2 | 18.8 | 9.5 | 12.2 | 5.8 |
| 5.0 | 0.9 | 0.7 | 0.8 | 0.8 | 0.1 |
| 6.6 | 1.1 | 0.8 | 0.4 | 0.8 | 0.3 |

48 Hours

| [X] | 1 | 2 | 3 | Mean (%) | SD × 100 |
|---|---|---|---|---|---|
| 0 | 100.0 | 100.0 | 100.0 | 100.0 | 0 |
| 0.16 | 59.1 | 66.9 | 71.5 | 65.9 | 6.3 |
| 0.33 | 57.9 | 66.2 | 79.1 | 67.7 | 10.7 |
| 1.6 | 52.9 | 47.2 | 35.0 | 45.0 | 9.1 |
| 3.3 | 8.2 | 18.8 | 9.5 | 12.2 | 5.8 |
| 5.0 | 0.9 | 0.7 | 0.8 | 0.8 | 0.1 |
| 6.6 | 1.1 | 0.8 | 0.4 | 0.8 | 0.3 |

72 Hours

| [X] | 1 | 2 | 3 | Mean (%) | SD × 100 |
|---|---|---|---|---|---|
| 0 | 100.0 | 100.0 | 100.0 | 100.0 | 0 |
| 0.16 | 62.4 | 64.2 | 75.7 | 67.5 | 7.2 |
| 0.33 | 61.5 | 69.3 | 77.2 | 69.4 | 7.9 |
| 1.6 | 79.0 | 62.9 | 30.7 | 57.6 | 24.6 |
| 3.3 | 0.7 | 0.3 | 0.3 | 0.4 | 0.3 |
| 5.0 | 0.8 | 0.1 | 0.3 | 0.4 | 0.4 |
| 6.6 | 0.5 | 0.3 | 0.7 | 0.5 | 0.2 |

Cell Morphology

24 Hours

| [X] | 1 | 2 | 3 | Mean |
|---|---|---|---|---|
| 0 | 5 | 5 | 5 | 5.0 |
| 0.16 | 5 | 5 | 5 | 5.0 |
| 0.33 | 4 | 4 | 4 | 4.0 |
| 1.6 | 3 | 2 | 2 | 2.3 |
| 3.3 | 1 | 1 | 1 | 1.0 |
| 5.0 | 1 | 1 | 1 | 1.0 |
| 6.6 | 1 | 1 | 1 | 1.0 |

48 Hours

| [X] | 1 | 2 | 3 | Mean |
|---|---|---|---|---|
| 0 | 5 | 5 | 5 | 5.0 |
| 0.16 | 5 | 5 | 5 | 5.0 |
| 0.33 | 4 | 3 | 5 | 4.0 |
| 1.6 | 3 | 3 | 2 | 2.7 |
| 3.3 | 1 | 1 | 1 | 1.0 |
| 5.0 | 1 | 1 | 1 | 1.0 |
| 6.6 | 1 | 1 | 1 | 1.0 |

72 Hours

| [X] | 1 | 2 | 3 | Mean |
|---|---|---|---|---|
| 0 | 5 | 5 | 5 | 5.0 |
| 0.16 | 5 | 5 | 5 | 5.0 |
| 0.33 | 5 | 5 | 5 | 5.0 |
| 1.6 | 3 | 3 | 2 | 2.7 |
| 3.3 | 1 | 1 | 1 | 1.0 |
| 5.0 | 1 | 1 | 1 | 1.0 |
| 6.6 | 1 | 1 | 1 | 1.0 |

### Example 12

### Polysorbate 80 (Tween 80)

ARPE-19 cells were incubated with increasing concentrations of the detergent Tween80® from 24 to 72 h, as described above.

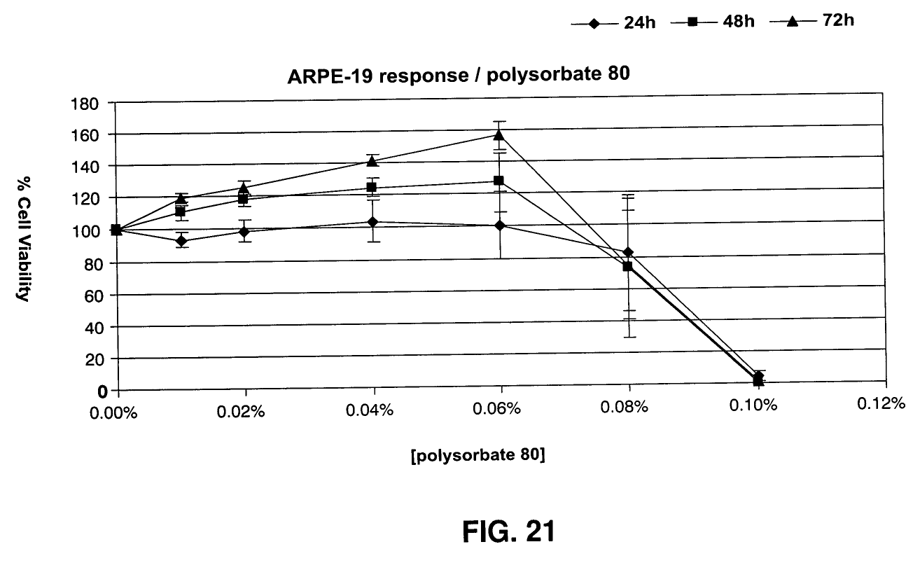

In a first approach (see Table 1), high doses of Tween80®, such as from 0.1% to 20%, were applied to ARPE-19 cells. Subsequently, the doses were decreased to a range from 0.01% to 0.1%, as 0.1% induced a dramatic decrease in viability of retinal cells. Results from the MTT assay are illustrated in FIG. 21.

It seemed that, as previously discussed for poloxamer 407 nf, the viability of cells increased at concentrations from 0.01% to 0.06%, then diminished to lethality at 0.1%. As discussed above, causes for the increase in cell proliferation were not considered in this study. Rather, it can be proposed that Tween80 is a surfactant that permeabilizes cell membranes, and therefore, Tween80® could possibly increase MTT crossing through the membrane into cells. Therefore, MTT conversion could be enhanced and MTT more rapidly converted into crystals, explaining intense coloration of cells. This situation might occur when cells are still able to convert MTT into crystals, until 0.06%. Raising concentrations (>0.08%) affect cell viability to such a point that even if MTT rapidly crossed the membrane towards mitochondria, it is no longer converted into formazan as viability diminishes and cells are less and less metabolicaly active. This interpretation correlates to the observation of cell morphology.

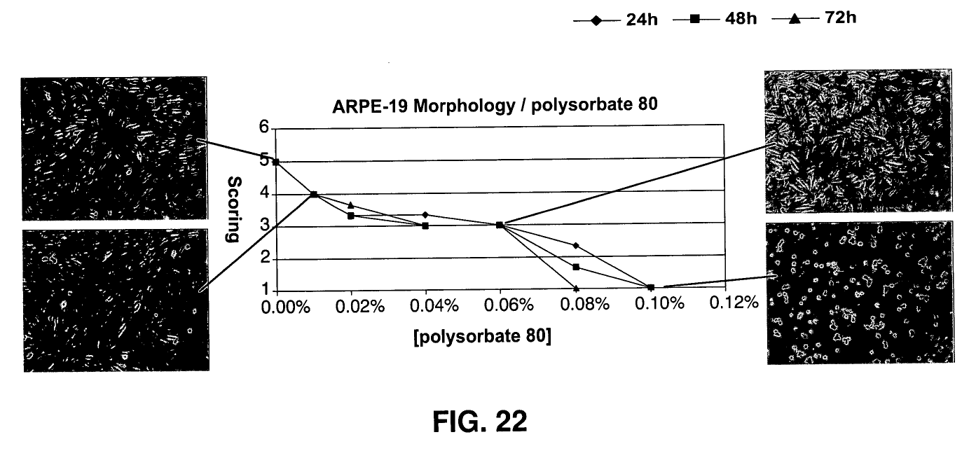

The cell morphology scoring for Tween80® is shown in FIG. 22. We can deduce that at concentrations as low as 0.01%, a perceptible change on cell aspect is noticeable, even after 24 h incubation. From 0.02 to 0.06% of Tween80®, cells appeared stressed with a rather long shape, but still form a confluent monolayer. At concentrations greater than 0.06%, the general aspect changed, for example, the cells appeared rather squared and non negligible proportion detached. At 0.1%, over 95% of the cells are floating in a typical round dead shape. From a morphological consideration, Tween80® appeared fairly aggressive to cells, as even low concentrations exerted an effect at minimum time of exposure (24 h). Cells exhibited an injured aspect from 0.06% which represents critical limit for lethality.

The IC₅₀ was not determined using the cell viability data, but based on the morphology data, the IC₅₀ could be estimated to be about 0.06%.

The raw data for cell viability and cell morphology are provided below

Cell Viability

24 Hours

| [Tween80] | | | | | |
|---|---|---|---|---|---|
| (%) | 1 | 2 | 3 | Mean (%) | SD × 100 |
| 0 | 100.0 | 100.0 | 100.0 | 100.0 | 0 |
| 0.01 | 90.3 | 99.4 | 90.9 | 93.5 | 5.1 |
| 0.02 | 91.8 | 98.2 | 105.8 | 98.6 | 7.0 |
| 0.04 | 91.8 | 102.8 | 117.5 | 104.0 | 12.9 |
| 0.06 | 83.6 | 93.8 | 124.4 | 100.6 | 21.2 |
| 0.08 | 60.3 | 63.0 | 123.7 | 82.3 | 35.8 |
| 0.1 | 1.8 | 1.7 | 8.1 | 3.9 | 3.6 |

48 Hours

| [Tween80] | | | | | |
|---|---|---|---|---|---|
| (%) | 1 | 2 | 3 | Mean (%) | SD × 100 |
| 0 | 100.0 | 100.0 | 100.0 | 100.0 | 0 |
| 0.01 | 108.9 | 107.2 | 115.5 | 110.5 | 4.4 |
| 0.02 | 121.7 | 116.1 | 115.7 | 117.8 | 3.4 |
| 0.04 | 125.9 | 118.6 | 130.7 | 125.1 | 6.1 |
| 0.06 | 117.8 | 116.1 | 148.5 | 127.5 | 18.2 |
| 0.08 | 40.5 | 56.0 | 122.3 | 72.9 | 43.4 |
| 0.1 | 0.5 | 0.9 | 0.2 | 0.5 | 0.4 |

72 Hours

| [Tween80] | | | | | |
|---|---|---|---|---|---|
| (%) | 1 | 2 | 3 | Mean (%) | SD × 100 |
| 0 | 100.0 | 100.0 | 100.0 | 100.0 | 0 |
| 0.01 | 122.1 | 119.6 | 117.7 | 119.8 | 2.2 |
| 0.02 | 128.9 | 127.3 | 121.3 | 125.9 | 4.0 |
| 0.04 | 138.4 | 145.8 | 140.5 | 141.6 | 3.8 |
| 0.06 | 147.7 | 157.0 | 165.5 | 156.8 | 8.9 |
| 0.08 | 58.4 | 52.1 | 113.9 | 74.8 | 34.0 |
| 0.1 | 0.8 | 0.7 | 0.3 | 0.6 | 0.3 |

Cell Morphology

24 Hours

| [Tween80] | | | | |
|---|---|---|---|---|
| (%) | 1 | 2 | 3 | Mean |
| 0 | 5 | 5 | 5 | 5.0 |
| 0.01 | 4 | 4 | 4 | 4.0 |
| 0.02 | 3 | 3 | 4 | 3.3 |
| 0.04 | 3 | 3 | 4 | 3.3 |
| 0.06 | 3 | 3 | 3 | 3.0 |
| 0.08 | 2 | 2 | 3 | 2.3 |
| 0.1 | 1 | 1 | 1 | 1.0 |

48 Hours

| [Tween80] | | | | |
|---|---|---|---|---|
| (%) | 1 | 2 | 3 | Mean |
| 0 | 5 | 5 | 5 | 5.0 |
| 0.01 | 4 | 4 | 4 | 4.0 |
| 0.02 | 3 | 3 | 4 | 3.3 |
| 0.04 | 3 | 3 | 3 | 3.0 |
| 0.06 | 3 | 3 | 3 | 3.0 |
| 0.08 | 2 | 2 | 1 | 1.7 |
| 0.1 | 1 | 1 | 1 | 1.0 |

72 Hours

| [Tween80] | | | | |
|---|---|---|---|---|
| (%) | 1 | 2 | 3 | Mean |
| 0 | 5 | 5 | 5 | 5.0 |
| 0.01 | 4 | 4 | 4 | 4.0 |
| 0.02 | 3 | 4 | 4 | 3.7 |
| 0.04 | 3 | 3 | 3 | 3.0 |
| 0.06 | 3 | 3 | 3 | 3.0 |
| 0.08 | 1 | 1 | 1 | 1.0 |
| 0.1 | 1 | 1 | 1 | 1.0 |

In summary, hydroxypropyl gamma-cyclodextrin (Cavasol) was less toxic to ARPE-19 cells than polysorbate 80. For example, 0.1% (w/v) of hydroxypropyl gamma-cyclodextrin resulted in only about a 10% or less reduction of cell survival at 24 hour, 48 hour, and 72 hour time points. At concentrations of 10% (w/v) of hydroxypropyl gamma-cyclodextrin, cell survival was about 80% of the initial value. In comparison to polysorbate 80, hydroxypropyl gamma-cyclodextrin exhibited substantially reduced toxicity relative to polysorbate 80 at tested concentrations from 0.1% (w/v) to 10% (w/v).

Sulfobutyl ether 4 beta-cyclodextrin (Captisol) was less toxic to ARPE-19 cells than polysorbate 80. For example, 0.1% (w/v) of sulfobutyl ether 4 beta-cyclodextrin resulted in only about a 10-20% reduction of cell survival at 24 hour, 48 hour, and 72 hour time points. At concentrations of 5% (w/v) of sulfobutyl ether 4 beta-cyclodextrin, cell survival was about 70-80% of the initial value. In comparison to polysorbate 80, sulfobutyl ether 4 beta-cyclodextrin exhibited substantially reduced toxicity relative to polysorbate 80 at tested concentrations from 0.1% (w/v) to 5% (w/v). In addition, at the 24 hour time point, sulfobutyl ether 4 beta-cyclodextrin at a concentration of 10% (w/v) resulted in about 50-60% cell survival, whereas polysorbate 80 had substantially zero cell survival at a concentration of 0.1% (w/v).

Hydroxypropyl beta-cyclodextrin (Kleptose) was less toxic to ARPE-19 cells than polysorbate 80. For example, 0.1% (w/v) of hydroxypropyl beta-cyclodextrin resulted in only about a 20-30% reduction of cell survival at 24 hour, 48 hour, and 72 hour time points. At concentrations of 1% (w/v) of hydroxypropyl beta-cyclodextrin, cell survival was about 70% of the initial value. In comparison to polysorbate 80, hydroxypropyl beta-cyclodextrin exhibited substantially reduced toxicity relative to polysorbate 80 at tested concentrations from 0.1% (w/v) to 1% (w/v). In addition, at the 24 hour time point, hydroxypropyl beta-cyclodextrin at a concentration of 5% (w/v) resulted in about 20% cell survival, whereas polysorbate 80 had substantially zero cell survival at a concentration of 0.1% (w/v).

The nonionic surfactant (Pluronic F127 Prill) was less toxic to ARPE-19 cells than polysorbate 80. For example, 0.1% (w/v) of Pluronic F127 Prill resulted in only about a 0-20% reduction of cell survival at 24 hour, 48 hour, and 72 hour time points. At concentrations of 1% (w/v) of Pluronic F127 Prill, cell survival was about 80-100% of the initial value. In comparison to polysorbate 80, Pluronic F127 Prill exhibited substantially reduced toxicity relative to polysorbate 80 at tested concentrations from 0.1% (w/v) to 1% (w/v).

In view of the above, hydroxypropyl gamma-cyclodextrin exhibited a lower toxicity compared to sulfobutyl ether 4-beta cyclodextrin, which exhibited a lower toxicity compared to hydroxypropyl beta-cyclodextrin, all of which exhibited a lower toxicity compared to polysorbate 80.

Overall results are summarized in Table 3, below.

TABLE 3

| | MTT | Morphology limit | Correlation |
|---|---|---|---|
| | IC₅₀ (%) | concentration (%) | between assays |
| Carboxymethylcellulose | ND | 0.8-1 | 0 |
| Hydroxypropylmethyl | ND | 0.8-1.2 | 0 |
| cellulose | | | |
| Poloxamer 407nf | ND | ND | +++ |
| Hyaluronic acid | 0.8-1.1 | 0.6-0.9 | ++ |
| Hydroxypropyl gamma- | ND | ND | ++ |
| CD | | | |
| Sulfobutyl ether 4beta- | 6.5-8.5 | 5.7-6.2 | + |
| CD | | | |
| Hydroxypropyl beta-CD | 2.2-2.5 | 2.6-3 | + |
| Benzyl alcohol | 0.07 | 0.07 | +++ |
| Borate buffer | ND | ND | +++ |
| Phosphate buffer | 1.3X-2X | 1.1X-1.4X | +++ |
| Polysorbate 80 | NA | 0.06 | NA |

NA: not applicable;
ND: not determined, experimental conditions do not allow to determine IC₅₀. “Correlation between assay” describes when cell viability data draw a parallel with cell morphology results.

The present results suggest that of 4 candidate viscosing agents (poloxamer 407 nf, CMC, HPMC and HA), poloxamer 407 nf appears to be less toxic, based on morphological consideration. For example, 24 h treatment shows almost no visible effect on cell shape. Carboxymethylcellulose (CMC) appeared to be well tolerated by cells in vitro, in our conditions. Both cell morphology and viability results showed scarce effect on cells (very moderate cytotoxicity). Hydroxypropylmethyl cellulose (HPMC) shows a different profile (particularly regarding mitochondrial metabolism) and was considered of moderate cytotoxicity. In these in vitro testing conditions, hyaluronic acid impacted cells the most, at concentrations over 0.6%. It was suspected that compared to other tested viscosing agents, highly viscous HA conditions, directly dispensed on cells in vitro, generate harsh environmental conditions, for example by blocking nutrient diffusion due to the stickiness of HA. However, it is noted that such situations may not occur in vivo after in intravitreal delivery of formulation since the RPE cell layer is located between Brüch's membrane and photoreceptor cells outer segment.

A substantial difference in behaviour between borate buffer and phosphate buffer towards ARPE-19 cells was observed. In our experimental conditions, borate buffer displayed discrete effects on cell metabolism, although provided significant changes in morphology even at the lowest doses tested. On the contrary, phosphate buffer decreased cell viability to a large extent upon increasing concentrations applied to cells. Cells were incubated with concentrations of phosphate buffer more than 6 times the one used in ophthalmic formulations. In Table 2, it can be seen that at the highest tested concentrations, osmolarity reaches values that affect cell integrity and is probably responsible for the damage measured on ARPE-19 cells (FIGS. 19-20). In comparision, cells were treated with concentrations under the concentrations of borate buffer prepared according the European Pharmacopeia.

The excipient, polysorbate 80, is barely tolerated by cells in vitro, probably due to membrane permeabilization which affect general cell metabolism and isotonicity. In addition, we noted possible cross-reaction between polysorbate 80 and the MTT assay reagent, and favor the idea that membrane disorganization enhances MTT penetration within the cells, when cells are still viable. After a certain concentration threshold, cell viability decreased as cells are no longer able to maintain active metabolism.

Benzyl alcohol is used as preservative in formulations. This excipient showed very aggressive impact on retinal cells. Even minimum doses as low as 0.05% had substantial measurable effects on cell viability and cell morphology. Benzyl alcohol is often used at concentrations as high as about 1% to prevent contamination of solution. It can be concluded that concentrations of benzyl alcohol less than 0.05% may be tolerated, but antimicrobial effectiveness may be limited.

Our results also demonstrate that hydroxypropyl beta-CD showed greater toxicity towards ARPE-19 cells than both sulfobutyl 4 ether beta-CD and hydroxy-propyl gamma-CD. Concentrations around 1% often used in ophthalmic formulations appeared of non-negligible effect, at least for sulfobutyl ether 4 beta-CD (6.5%<IC₅₀<8.5%) and hydroxypropyl beta-CD (IC₅₀=2%) on cell metabolism even if cell phenotype did not markedly vary. Under these test conditions, 10% of hydroxypropyl gamma-CD showed no measurable effect on both cell parameters. Therefore hydroxypropyl gamma-CD may provide substantial advantages for drug delivery systems for intravitreal administration.

The methods described herein can be used to screen additional excipient agents for use in the present drug delivery systems. In view of the disclosure herein, such methods are routine to persons of ordinary skill in the art. Excipients with reduced toxicity, alone or in combination with other excipients, are selected for the present drug delivery systems so that administration of the drug delivery system to the eye does not cause substantial undersirable effects.

### Examples 13 to 16

Four compositions are as follows:

| Ingredient | Example 13 | Example 14 | Example 15 | Example 16 |
|---|---|---|---|---|
| Triacinolone | 2% (w/v) | 2% (w/v) | 4% (w/v) | 4% (w/v) |
| acetonide | | | | |
| Sodium | 0.05% (w/v) | 0.5% (w/v) | 0.05% (w/v) | 0.5% (w/v) |
| Hyaluronate | | | | |
| (0.6 × 10⁶ | | | | |
| DALTONS) | | | | |
| Sodium | 0.4% (w/v) | 0.4% (w/v) | 0.4% (w/v) | 0.4% (w/v) |
| Phosphate | | | | |
| Vitamin | 0.5% (w/v) | 0.5% (w/v) | 0.0 | 0.0 |
| E-TPGS | | | | |
| gamma- | 0.5% (w/v) | 0.5% (w/v) | 0.0 | 0.0 |
| cyclodextrin | | | | |
| Water for | q.s. | q.s. | q.s. | q.s. |
| Injection | | | | |
| Viscosity at | 20 cps | 500 cps | 20 cps | 500 cps |
| shear rate | | | | |
| 0.1/second | | | | |

Each of these compositions is prepared as follows.

A concentrated triamcinolone acetonide dispersion is made by combining triamcinolone acetonide with water, Vitamin E-TPGS and gamma-cyclodextrin, if any. These ingredients are mixed to disperse the triamcinolone acetonide, and then autoclaved. The sodium hyaluronate may be purchased as a sterile powder or sterilized by filtering a dilute solution followed by lyophylization to yield a sterile powder. The sterile sodium hyaluronate is dissolved in water to make an aqueous concentrate. The concentrated triamcinolone acetonide dispersion is mixed and added as a slurry to the sodium hyaluronate concentrate. Water is added q.s. and the mixture is mixed until homogenous.

Triamcinolone acetonide in each of these compositions can be easily re-suspended by gentle inversion. These compositions can be marketed in small volume pharmaceutical grade glass bottles, and are found to be therapeutically effective against macular edema when injected intravitreally into human eyes.

### Example 17

### Use of an Ophthalmic Composition Containing a Retinal Friendly Excipient To Treat Neovascularization

A 68 year old female complains to her physician that it is becoming difficult to see. The physician determines that her retinas are exhibiting neovascularization. A composition containing 5% (w/v) of a triamcinolone acetonide and 0.5% (w/v) hydroxypropyl gamma-cyclodextrin is injected in the vitreous of both of the woman's eyes using a trocar. After a single injection, neovascularization is halted, and the patient receives additional injections as prescribed by her physician.

### Example 18

Example 7 is repeated by administering compositions containing dexamethasone and prednisolone instead of triamcinolone acetonide. Similar results are obtained.

### Example 19

Example 7 is repeated by administering compositions containing other excipient agents in amounts that are not substantially toxic to retinal pigment epithelial cells (e.g., result in less than 50% cell death when tested in vitro, as described in the examples). Beneficial results are obtained with each of the different compositions.

### Example 20

### In vivo Testing of Ophthalmic Compositions

Compositions of the present invention were also administered to living animals to evaluate the ocular effects of the compositions. A single intravitreal injection of one of many different ophthalmic formulations was administered to female New Zealand White (NZW) rabbits. The rabbits, including the eyes of the rabbits, were observed for a period of time of up to about 3 months.

Eight groups of rabbits (3/group) were given a single intravitreal injection (0.1 mL) of one of the following compositions into the left eye of a rabbit:
(1) Kenalog-40 (4% triamcinolone acetonide (TA); 4 mg TA/0.1 mL)
(2) 2% hyaluronic acid (HA)+4% TA
(3) 0.5% sulfobutyl ether beta-cyclodextrin+4% TA
(4) 5% sulfobutyl ether beta-cyclodextrin+4% TA
(5) 0.5% gamma-cyclodextrin+4% TA
(6) 5% gamma-cyclodextrin+4% TA
(7) 0.5% vitamin E-tocopheryl polyethyleneglycol succinate (TPGS)+4% TA
(8) 2% vitamin E-TPGS+4% TA.

The right eye of the rabbit received a similar volume of 0.9% sodium chloride. Rabbits were sacrificed and enucleated at week 7 (1/group) or at 3 months (2/group).

Evaluated parameters included viability, clinical observations, gross ocular observations, slit lamp biomicroscopy, including pupillary reflex, ophthalmoscopy, electroretinography (ERG), intraocular pressure (IOP), body weight, macroscopic observations, and microscopic pathology (ocular tissues).

Mortality of the rabbits was not observed. In addition, there were no detectable differences between experimental eyes and control eyes based on clinical observations, IOP, body weight, or macroscopic observations. Up to one week post injection, both experimental and control eyes exhibited one or more of congestion, swelling, ocular discharge, and tearing, which appears to be related to the intravitreal injection. Such ocular irritations typically resolved within 2-3 weeks after the injection. Incomplete pupillary responses were observed in eyes given composition (8) above. Small particles were observed intermittently in the vitreous with all drug formulations. One rabbit was sacrificed on day 13 due to severe ocular reactions resulting from the intravitreal injection.

Decreased ERG b-wave (greater than or equal to 30% relative to baseline values and/or values obtained from the contralateral control eye) were observed in eyes given compositions (3), (4), (5), and (8), above. Compositions 3 and 4 resulted in values of about 39% to 67% at 3 months post-injection. Composition 5 resulted in values of about 42% to 62% at 3 months post-injection. Composition 8 resulted in values of about 30% to about 65% at 1 week, 2 weeks, and 3 months post injection. No significant changes in the ERG b-wave were observed in eyes given compositions (1) and (2).

One eye given composition (5) exhibited minimal subacute vitreitis at week 7 post injection. Another eye given composition (6) exhibited chronic chorioretinitis at week 7 post injection. One eye given composition (4) exhibited minimal neutrophil infiltration of the choroid at 3 months post-injection. Degenerative and necrotic lesions of the optic nerve head and retina characterized by edema, axonal eosinophilia, and scarring were observed in eyes given compositions (7) and (8).

These results in combination with the in vitro data describe methods for screening excipient agents that have a relatively low toxicity, and can be used in developing compositions that are suitable for intraocular administration to the eye.

Although the present invention has been described in detail with regard to certain preferred compositions and methods, other embodiments, versions, and modifications within the scope of the present invention are possible. For example, combination therapies are also provided with the present compositions. As one example, the present compositions may comprise a combination of an anti-inflammatory agent, such as a steroid, and an intraocular pressure reducing agent, such as an alpha-2-adrenergic agonist, to reduce inflammation and intraocular pressure substantially at the same time. Another example, includes a composition comprising an anti-excitotoxic agent which may be used as a neuroprotectant, and an anti-inflammatory agents. Combination therapies may use any and all possible combinations of therapeutic agents disclosed herein so long as such combinations are not mutually exclusive.

The present invention also includes within its scope the use of a therapeutic component, such as one or more therapeutic agents, and one or more cyclodextrins in the preparation of a medicament for the treatment of an ocular condition, such as a disease or disorder of the poterior segment of an eye, by administration of the medicament to the interior of an eye.

All references, articles, patents, applications and publications set forth above are incorporated herein by reference in their entireties.

While this invention has been described with respect to various specific examples and embodiments, it is to be understood that the invention is not limited thereto and that it can be variously practiced within the scope of the following claims.

1. A therapeutic ophthalmic composition useful for injection into a posterior segment of an eye of an individual, comprising
a therapeutic component present in an amount effective in providing a desired therapeutic effect to an individual when the composition is administered to the interior of an eye of the individual; and
an excipient component comprising an excipient agent other than polysorbate 80 or benzyl alcohol, the excipient component present in an amount that is less toxic to retinal pigment epithelial cells relative to an equal amount of an excipient selected from the group consisting of polysorbate 80 and benzyl alcohol.

2. The composition of claim 1, wherein the therapeutic component comprises at least one therapeutic agent selected from the group consisting of steroids and steroid precursors.

3. The composition of claim 1, wherein the therapeutic component comprises at least one steroid selected from the group consisting of cortisone, dexamethasone, prednisolone, prednisolone acetate, triamcinolone, and triamcinolone acetonide.

4. The composition of claim 1, wherein the excipient component comprises a cyclodextrin present in an amount from about 0.1% (w/v) to about 5% (w/v).

5. The composition of claim 1, wherein the excipient component comprises at least one cyclodextrin selected from the group consisting of alpha-cyclodextrins, alpha-cyclodextrin derivatives, beta-cyclodextrins, beta-cyclodextrin derivatives, gamma-cyclodextrins, and gamma-cyclodextrin derivatives.

6. The composition of claim 1, wherein the excipient component consists of at least one cyclodextrin selected from the group consisting of sulfobutyl ether 4-beta-cyclodextrin, hydroxypropyl beta-cyclodextrin, and hydroxypropyl gamma-cyclodextrin.

7. The composition of claim 1, wherein the excipient component comprises an amount of hydroxypropyl gamma-cyclodextrin from about 0.1% (w/v) to about 10% (w/v).

8. The composition of claim 1, wherein the excipient component comprises an amount of sulfobutyl ether 4-beta-cyclodextrin from about 0.1% (w/v) to about 10% (w/v).

9. The composition of claim 1, wherein the excipient component comprises an amount of hydroxypropyl beta-cyclodextrin from about 0.1% (w/v) to about 5% (w/v).

10. The composition of claim 1, further comprising an ophthalmically acceptable aqueous based vehicle component suitable for administration to the interior of the eye.

11. The composition of claim 1, wherein the excipient component comprises at least one excipient agent selected from the group consisting of sulfobutyl ether 4 beta cyclodextrin, hydroxypropyl beta-cyclodextrin, hydroxypropyl gamma-cyclodextrin, carboxymethylcellulose, hydroxypropylmethyl cellulose, and boric acid.

12. A therapeutic ophthalmic composition useful for injection into a posterior segment of an eye of an individual, comprising
a therapeutic component present in an amount effective in providing a desired therapeutic effect to an individual when the composition is administered to the interior of an eye of the individual; and
an excipient component effective in reducing the toxicity of the ophthalmic composition to retinal pigment epithelial cells of the eye of the individual relative to a second substantially identical composition which comprises at least one excipient selected from the group consisting of polysorbate 80 and benzyl alcohol, when the ophthalmic composition is administered to the interior of the eye of the individual.

13. The composition of claim 12, wherein the therapeutic component comprises a therapeutic agent selected from the group consisting of steroids and steroid precursors, and the cyclodextrin component comprises at least one cyclodextrin selected from the group consisting of sulfobutyl ether 4-beta-cyclodextrin, hydroxypropyl beta-cyclodextrin, and hydroxypropyl gamma-cyclodextrin.

14. A therapeutic ophthalmic composition useful for injection into a posterior segment of an eye of an individual, comprising
a therapeutic component present in an amount effective in providing a desired therapeutic effect to an individual when the composition is administered to the interior of an eye of the individual; and
a cyclodextrin component in an amount from about 0.5% (w/v) to about 5.0% (w/v) of the composition and effective in solubilizing a therapeutic agent of the therapeutic component.

15. The composition of claim 14, wherein the therapeutic component comprises a corticosteroid, and the cyclodextrin component is effective in solubilizing less than 50% of the corticosteroid.

16. A method for treating a posterior segment ocular condition, comprising administering the composition of claim 1 into the interior of an eye of an individual.

17. The method of claim 16, wherein the administering comprises intravitreally injecting the composition into the eye of the individual.

18. The method of claim 16, wherein the administering comprises at least one injecting step selected from the group consisting of suprachoroidal injecting, subretinal injecting, subtenon's injecting, pars-plana injecting, retrobulbar injecting, and intrascieral injecting.

19. A method for treating a posterior segment ocular condition, comprising administering the composition of claim 12 into the interior of an eye of an individual.

20. A method for treating a posterior segment ocular condition, comprising administering the composition of claim 14 into the interior of an eye of an individual.

21. A method for producing the composition of claim 1, comprising selecting an amount of the excipient component to include in the composition based on data obtained from contacting cultured retinal pigment epithelial cells with the excipient component.
